SEQUENCE LISTING (43 sequences)

SEQ ID NO: 1 - RNA, 287 nt, Teschovirus A
uacuugguuaugaauucauuguauuaaccccucugaaagaccugcucuggcgcgagcuaaagcgcaauugucaccagguauugcaccaaugguggcgacaggguacagaagagcaaguacuccugacuggguaaugggacugcauugcauaucccuaggcaccuauugagauuucucuggggcccaccagcguggaguuccuguaugggaaugcaggacuggacuugugcugccugacagggucgcggcuggccgucuguacuuuguauagucaguugaaacucacc

SEQ ID NO: 2 - DNA, 302 bp, Hepacivirus C
tcccctgtgaggaactactgtcttcacgcagaaagcgtctagccatggcgttagtatgagtgtcgtgcagcctccaggcccccccctcccgggagagccatagtggtctgcggaaccggtgagtacaccggaattgccaggacgaccgggtcctttcttggatcaatcccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcacc

SEQ ID NO: 3 - DNA, 463 bp, Cardiovirus A
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 4 - DNA, 204 bp, Cricket paralysis virus
agcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacctgctacatttcaaga

SEQ ID NO: 5 - DNA, 332 bp, Artificial Sequence
  Description of Artificial Sequence Synthetic oligonucleotide
  Description of Combined DNA/RNA Molecule Synthetic oligonucleotide
tgacagcccacatggcattccacttatcactggcatccttttattuacuugguuaugaauucauuguauuaaccccucugaaagaccugcucuggcgcgagcuaaagcgcaauugucaccagguauugcaccaaugguggcgacaggguacagaagagcaaguacuccugacuggguaaugggacugcauugcauaucccuaggcaccuauugagauuucucuggggcccaccagcguggaguuccuguaugggaaugcaggacuggacuugugcugccugacagggucgcggcuggccgucuguacuuuguauagucaguugaaacucacc

SEQ ID NO: 6 - DNA, 40 bp, Artificial Sequence
  Description of Artificial Sequence Synthetic oligonucleotide
ggtggctttaccaacagtaccggattgccaagcttgggct

SEQ ID NO: 7 - DNA, 40 bp, Artificial Sequence
  Description of Artificial Sequence Synthetic oligonucleotide
cgctgggcccttcttaatgtttttggcatcttccatggtg

SEQ ID NO: 8 - DNA, 40 bp, Artificial Sequence
  Description of Artificial Sequence Synthetic oligonucleotide
atggcgctgggcccttcttaatgtttttggcatcttccat

SEQ ID NO: 9 - DNA, 40 bp, Artificial Sequence
  Description of Artificial Sequence Synthetic oligonucleotide
ggtagaatggcgctgggcccttcttaatgtttttggcatc

SEQ ID NO: 10 - DNA, 40 bp, Artificial Sequence
  Description of Artificial Sequence Synthetic oligonucleotide
caggtcgactctagactcgaggctagcgagctcgtttaaa

SEQ ID NO: 11 - DNA, 40 bp, Artificial Sequence
  Description of Artificial Sequence Synthetic oligonucleotide
gctcagcggtggcagcagccaactcagcttcctttcgggc

SEQ ID NO: 12 - DNA, 40 bp, Artificial Sequence
  Description of Artificial Sequence Synthetic oligonucleotide
tcatgtctgctcgaagcggccgccccaaggggttatgcta

SEQ ID NO: 13 - DNA, 40 bp, Artificial Sequence
  Description of Artificial Sequence Synthetic oligonucleotide
ccacaggcggaggcgaaagcagcccggacagctgaggccg

SEQ ID NO: 14 - DNA, 40 bp, Artificial Sequence
  Description of Artificial Sequence Synthetic oligonucleotide
caaggagcaccttcatgttgcgttcggagaggcgcagcat

SEQ ID NO: 15 - DNA, 40 bp, Artificial Sequence
  Description of Artificial Sequence Synthetic oligonucleotide
cctgcacagacggtcactcaaagaaagatgtccctgtgcc

SEQ ID NO: 16 - DNA, 40 bp, Artificial Sequence
  Description of Artificial Sequence Synthetic oligonucleotide
gaatgtgaggggagtgggtccgctccaccagatgccagca

SEQ ID NO: 17 - DNA, 40 bp, Artificial Sequence
  Description of Artificial Sequence Synthetic oligonucleotide
agagcgggcctttgaggccctcgcgcttccaggactgcag

SEQ ID NO: 18 - DNA, 40 bp, Artificial Sequence
  Description of Artificial Sequence Synthetic oligonucleotide
ggggggcagggggcggccagggtatgtacatgaggaggtg

SEQ ID NO: 19 - DNA, 40 bp, Artificial Sequence
  Description of Artificial Sequence Synthetic oligonucleotide
atggcaacatgcactggaagctcctcgtggcggaccatcc

SEQ ID NO: 20 - DNA, 40 bp, Artificial Sequence
  Description of Artificial Sequence Synthetic oligonucleotide
ttgtcggggtccacgcaatctacacatggtcgcaaggtct

SEQ ID NO: 21 - DNA, 40 bp, Artificial Sequence
  Description of Artificial Sequence Synthetic oligonucleotide
attcccagggcctccccgcggccccgcaggggagggaggc

SEQ ID NO: 22 - DNA, 45 bp, Artificial Sequence
  Description of Artificial Sequence Synthetic oligonucleotide
tgacagcccacatggcattccacttatcactggcatccttttatt

SEQ ID NO: 23 - DNA, 199 bp, Acute bee paralysis virus
cacaacatggttacccatagattgaggaaatttccaataaactcagtattaaggcttgttgtgttggacaaggtgccctatttagggtgaggagccttactggcagccccagtgaatcctccattggataggaacagctatattgggtagttgtagcagttgtattcaaatgaatgcagcgttccgaaatatcatacct

SEQ ID NO: 24 - DNA, 494 bp, Tremovirus A
tttgaaagaggcctccggagtgtccggaggctctctttcgacccaacccatactggggggtgtgtgggaccgtacctggagtgcacggtatatatgcattcccgcatggcaagggcgtgctaccttgccccttgacgcatggtatgcgtcatcatttgccttggttaagccccatagaaacgaggcgtcacgtgccgaaaatccctttgcgtttcacagaaccatcctaaccatgggtgtagtatgggaatcgtgtatggggatgattaggatctctcgtagagggataggtgtgccattcaaatccagggagtactctggctctgacattgggacatttgatgtaaccggacctggttcagtatccgggttgtcctgtattgttacggtgtatccgtcttggcacactgaaagggtatttttgggtaatcctttcctactgcctgatagggtggcgtgcccggccacgagagattaagggtagcaatttaaac

SEQ ID NO: 25 - DNA, 184 bp, Aphid lethal paralysis virus
aattactaatttgatctttaggttataatgttaggactataaaaattagcttaatgcatttagtaatttaaggcttagttatttaactttacttatcaagatggccgttggcagccccacgaaatctagattagtccgaatgtcctattttgattaggtggtcagataggtcagaaactcacct

SEQ ID NO: 26 - DNA, 190 bp, Black queen cell virus
ccaacaatgtgatcttgcttgcggaggcaaaatttgcacagtataaaatctgcaagtagtgctattgttggaatcaccgtacctatttaggtttacgctccaagatcggtggatagcagccctatcaatatctaggagaactgtgctatgtttagaagattaggtagtctctaaacagaacaatttacct

SEQ ID NO: 27 - DNA, 900 bp, Pestivirus A
gtatacgagaattagaaaaggcactcgtatacgtattgggcaattaaaaataataattaggcctagggaacaaatccctctcagcgaaggccgaaaagaggctagccatgcccttagtaggactagcataatgaggggggtagcaacagtggtgagttcgttggatggcttaagccctgagtacagggtagtcgtcagtggttcgacgccttggaataaaggtctcgagatgccacgtggacgagggcatgcccaaagcacatcttaacctgagcgggggtcgcccaggtaaaagcagttttaaccgactgttacgaatacagcctgatagggtgctgcagaggcccactgtattgctactaaaaatctctgctgtacatggcacatggagttgatcacaaatgaacttttatacaaaacatacaaacaaaaacccgtcggggtggaggaacctgtttatgatcaggcaggtgatcccttatttggtgaaaggggagcagtccaccctcaatcgacgctaaagctcccacacaagagaggggaacgcgatgttccaaccaacttggcatccttaccaaaaagaggtgactgcaggtcgggtaatagcagaggacctgtgagcgggatctacctgaagccagggccactattttaccaggactataaaggtcccgtctatcacagggccccgctggagctctttgaggagggatccatgtgtgaaacgactaaacggatagggagagtaactggaagtgacggaaagctgtaccacatttatgtgtgtatagatggatgtataataataaaaagtgccacgagaagttaccaaagggtgttcaggtgggtccataataggcttgactgccctctatgggtcacaacttgctcagacacgaa

SEQ ID NO: 28 - DNA, 148 bp, Tobamovirus sp.
gaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaa

SEQ ID NO: 29 - DNA, 376 bp, Artificial Sequence
  Description of Artificial Sequence Synthetic polynucleotide
gcatacgaggttagttcattctcgtatacacgattggacaaatcaaaattataatttggttcagggcctccctccagcgacggccgaactgggctagccatgcccatagtaggactagcaaaacggagggactagccatagtggcgagctccctgggtggtctaagtcctgagtacaggacagtcgtcagtagttcgacgtgagcagaagcccacctcgagatgctacgtggacgagggcatgccaagacacaccttaaccctagcgggggtcgctagggtgaaatcacaccacgtgatgggagtacgacctgatagggcgctgcagaggcccactattaggctagtataaaaatctctgctgtacatggcacatg

SEQ ID NO: 30 - DNA, 189 bp, Drosophila C virus
gttaagatgtgatcttgcttccttatacaattttgagaggttaataagaaggaagtagtgctatcttaataattaggttaactatttagttttactgttcaggatgcctattggcagccccataatatccaggacaccctctctgcttcttatatgattaggttgtcatttagaataagaaaataacct

SEQ ID NO: 31 - DNA, 694 bp, Triatoma virus
caaaattgcgtgcgagaaagcacgcaaatcaaagtctagtgcgtaattcactcactaccggcgtaatttgtggttatgctattgcgttgagagtgttgtgggcgtagtacgggggcttttggtttgtgtgtgattaatatgcattcccagttttgtttagttttagattactgatttatttttcgaactacccgaatttatttaggctcttcgagaaataatgatgaactgtcctcaacaagtatgaaaagcaattatttgtgaagttacttgttgttgtaagattattgacctcttagatttttctaagttgtaatgctttgttttctgattgactagattatgaatccaattaaaaggagtagtggtctaatatagtctgtgtgacctgcaggcattttgtgaaaagggtaaagtatgaaagctactctcagaaaagtacttatgtattgatgagagccttaaaatgactttatattcacaaaactgctggaagacaatgatctggggtatcacattccctcttaggttaagtttcgcactactaggaatttttgcgaaaattcttaattccattcttatggtgtagtgtttgttcttttcaattggggcgtatctctctttcgcagagaccctggcccaaccctgaaatgaataaaattttacaaaaattcatcgaaaatcgaccttctag

SEQ ID NO: 32 - DNA, 760 bp, Artificial Sequence
  Description of Artificial Sequence Synthetic polynucleotide
attgatgtgttggtcgtttgccaatcggagggcgacaggtcgtttgccaatcggagggcgacaggcacaggtcgctccgagttctagtagtgtgggaacttgttactactgatgaaacgaggtagtgacactgactacctgcgaacgaggtcggggccctcccttcttccttcacccaactttcacttttcgttccactttagcaggggtcttctttctatccccctggcggcattggaactagccgtcgcgtcttaacgcgcagccctgaaggccccacaccttgtggatcttgccgtgggtatgtttctggcatgtgtttctcaagcctgcaaccgaagccgaacagccacatgaacagtttgagcgtggtagcgctgtgtgagttggcggtggatccccctcgtggtaacacgagcccccgtggccaaaagcccagtgtttacagcacctctcacatccaggacgaccccatcctggcgctcactcttagtagtatggcttagtacgcattaggtggtaagccgagctctccctcggccttgttctgaatgcacacatgtctaggggctaaggatgtcctacaggtacccgcacgtaaccttcagagagtgcggatctgagtaggagaccgtggtgcactgctttacagatgcagccccggtttaaaaagcgtctatgcccctacagggtagcggtgggccgcgccctttccttttaaaactacttgttctatggtgacaatggcaggaaacatgat

SEQ ID NO: 33 - DNA, 531 bp, Cardiovirus A
cggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccaccgggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaagcgtctaggccccccgaaccacggggacgtggttttcctttgaaaaacacgatgataatatggccacaacc

SEQ ID NO: 34 - DNA, 579 bp, Rhopalosiphum padi virus
gataaaagaacctataatcccttcgcacaccgcgtcacaccgcgctatatgctgctcattaggaattacggctccttttttgtggatacaatctcttgtatacgatatacttattgttaatttcattgacctttacgcaatcctgcgtaaatgctggtatagggtgtacttcggatttccgagcctatattggttttgaaaggacctttaagtccctactatactacattgtactagcgtaggccacgtaggcccgtaagatattataactattttattatattttattcaccccccacattaatcccagttaaagctttataactataagtaagccgtgccgaaacgttaatcggtcgctagttgcgtaacaactgttagtttaattttccaaaatttatttttcacaatttttagttaagattttagcttgccttaagcagtctttatatcttctgtatattattttaaagtttataggagcaaagttcgctttactcgcaatagctattttatttattttaggaatattatcacctcgtaattatttaattataacattagctttatctatttata

SEQ ID NO: 35 - DNA, 852 bp, Hepatovirus A
tccctcttggaagtccctggtgaggggacttgatacctcaccgccgtttgcctaggctataggctaaattttccctttcccttttccctttcccattcccttttgcttgtaaatattgattcctgcaggttcagggttcttaaatctgtttctctataagaacactcattttcacgctttctgtcttctttcttccagggctctccccttgccctaggctctggccgttgcgcccggcggggtcaactccatgattagcatggagctgtaggagtctaaattggggacacagatgtttggaacgtcaccttgcagtgttaacttggctttcatgaatctctttgatcttccacaaggggtaggctacgggtgaaacctcttaggctaatacttctatgaagagatgccttggatagggtaacagcggcggatattggtgagttgttaagacaaaaaccattcaacgccggaggactgactctcatccagtggatgcattgagtggattgactgtcagggctgtctttaggcttaattccagacctctctgtgcttagggcaaacatcatttggccttaaatgggattctgtgagaggggatccctccattgacagctggactgttctttggggccttatgtggtgtttgcctctgaggtactcaggggcatttaggtttttcctcattcttaaataataatgaacatgtctagacaaggtattttccagactgttgggagtggtcttgaccacatcctgtctttggcagacattgaggaagagcaaatgattcaatcagttgataggactgcagtgactggtgcttcttattttacttctgtggatcaatct

SEQ ID NO: 36 - DNA, 50 bp, Artificial Sequence
  Description of Artificial Sequence Synthetic oligonucleotide
caggtcgactctagactcgaggctagcgagctcgtttaaacaactagaat

SEQ ID NO: 37 - DNA, 40 bp, Artificial Sequence; identical to SEQ ID NO: 10
  Description of Artificial Sequence Synthetic oligonucleotide

SEQ ID NO: 38 - DNA, 30 bp, Artificial Sequence
  Description of Artificial Sequence Synthetic oligonucleotide
cgactctagactcgaggctagcgagctcgt

SEQ ID NO: 39 - DNA, 20 bp, Artificial Sequence
  Description of Artificial Sequence Synthetic oligonucleotide
ctagactcgaggctagcgag

SEQ ID NO: 40 - DNA, 15 bp, Artificial Sequence
  Description of Artificial Sequence Synthetic oligonucleotide
ctcgaggctagcgag

SEQ ID NO: 41 - DNA, 635 bp, Unknown
  Description of Unknown PV-IRES sequence
gacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttggttagcactcaaccccagagtgtagcttaggctgatgagtctggacatccctcaccggtgacggtgttccaggctgcgttggcggcctacctatggctaacgcatgggacgctagttgtgaacaaggtgtgaagagcctattgagctacataagaatcctccggcccctgaatgcggctaatcccaacctcggagcaggtggttcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatg

SEQ ID NO: 42 - DNA, 458 bp, Unknown
  Description of Unknown FMDV-IRES sequence
cacgatttaagcaggtttccacaactgataaaactcgtgcaacttgaaactccgcctggtctttccaggtctagaggggttacactttgtactgtgctcgactccacgcccggtccactggcgggtgttagtagcagcactgttgtttcgtagcggagcatggtggccgtgggaactcctccttggtgacaagggcccacggggccgaaagccacgtccagacggacccaccatgtgtgcaaccccagcacggcaacttttactgcgaacaccaccttaaggtgacactggtactggtactcggtcactggtgacaggctaaggatgcccttcaggtaccccgaggtaacacgggacactcgggatctgagaaggggattgggacttctttaaaagtgcccagtttaaaaagcttctacgcctgaataggcgaccggaggccggcgcctttccattac

SEQ ID NO: 43 - DNA, 352 bp, Unknown
  Description of Unknown FMDV-IRES sequence
caacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagct

(19) United States
(12) Patent Application Publication
(10) Pub. No.: US 2023/0090706 A1
(43) Pub. Date: Mar. 23, 2023

(54) METHODS AND COMPOSITIONS COMPRISING TRANS-ACTING TRANSLATIONAL ACTIVATORS

(71) Applicant: The University of Chicago, Chicago, IL (US)

(72) Inventors: Bryan DICKINSON, Chicago, IL (US); Yang CAO, Chicago, IL (US)

(73) Assignee: The University of Chicago, Chicago, IL (US)

(21) Appl. No.: 17/905,116

(22) PCT Filed: Feb. 26, 2021
(86) PCT No.: PCT/US2021/070200

§ 371 Date: Aug. 26, 2022

Related U.S. Application Data
(60) Provisional application No. 62/993,151, filed on Mar. 23, 2020.
(60) Provisional application No. 62/983,030, filed on Feb. 28, 2020.

(51) Int. Cl. C12N 15/113; C12N 15/67

(52) U.S. Cl. CPC C12N 15/113; C12N 15/67

(57) ABSTRACT

The current disclosure relates to nucleic acid therapeutics that target mRNA molecules and recruit translation machinery to increase the translation from the mRNA, thus increasing the protein product in a subject or cell. Accordingly, aspects of the disclosure relate to a chimeric nucleic acid comprising a targeting region and a translational activating region, wherein the translational activating region comprises at least one ribosome and/or translation factor binding site and wherein the targeting region comprises a region that is complementary to a target mRNA.

### CROSS-REFERENCE TO RELATED APPLICATIONS

This application claims the benefit of priority of U.S. Provisional Patent Application No. 62/993,151 filed Mar. 23, 2020, and U.S. Provisional Patent Application No. 62/983,030 filed Feb. 28, 2020, all of which are hereby incorporated by reference in their entirety.

### BACKGROUND OF THE INVENTION

This invention was made with government support under GM119840 awarded by the National Institutes of Health. The government has certain rights in the invention.

### FIELD OF THE INVENTION

This invention relates to the field of molecular biology and medicine.

### II. BACKGROUND

RNA molecule regulation has emerged as an important therapeutics target and tool to control gene expression. For example, the first RNAi-based drug, patisiran, was approved in August 2018 for treating hereditary transthyretin amyloidosis, marking a new era for RNA therapeutics. Just one year later, the second-ever RNAi drug, givosiran, was approved. Besides the huge progress of RNAi, more diverse RNA therapies are being developed by companies including Moderna, Stoke Therapeutics, et al. to develop mRNA vaccines and to treat haploinsufficiency diseases. RNA therapies enable one to target traditional “undruggable” targets without permanently altering the genome, and its programmability make treatment cost-effective and easy to combine with other drugs. There is a need in the art for the development of additional RNA therapeutics that can target diseases that are otherwise untreatable through traditional therapeutic approaches.

### SUMMARY OF THE INVENTION

The current disclosure relates to nucleic acid therapeutics that target mRNA molecules and recruit translation machinery to increase the translation from the mRNA, thus increasing the protein product in a subject or cell. Accordingly, aspects of the disclosure relate to a chimeric nucleic acid comprising a targeting region and a translational activating region, wherein the translational activating region comprises at least one ribosome and/or translation factor binding site and wherein the targeting region comprises a region that is complementary to a target mRNA. Further aspects relate to a method for increasing translation of a target mRNA in a cell comprising administering the nucleic acid of the disclosure, wherein the target region of the nucleic acid is complementary to the target mRNA. Yet further aspects relate to a method for treating a haploinsufficiency disorder in a subject, wherein the haploinsufficiency disorder is further defined as a deficiency in the protein expression of one or both alleles of a target gene, the method comprising administering a nucleic acid of the disclosure to the subject, wherein the target region of the nucleic acid is complementary to a mRNA transcribed from the target gene. The disclosure also provides method for treating a disease in a subject comprising administering a nucleic acid of the disclosure. Further aspects relate to a method for treating cancer in a subject comprising administering a nucleic acid of the disclosure to the subject. Also disclosed are cDNAs encoding the nucleic acid of the disclosure, vectors comprising the cDNAs, and host cells comprising the nucleic acids, vectors, or cDNAs of the disclosure.

In some embodiments, the nucleic acid is a single-stranded nucleic acid. In some embodiments, the nucleic acid is a double-stranded nucleic acid. In some embodiments, the mRNA comprises a mammalian mRNA. In some embodiments, the mRNA comprises or corresponds to a mRNA produced by a human, mouse, dog, cat, pig, rat, rabbit, eukaryotic, or prokaryotic cell. In some embodiments, the mRNA comprises a bacterial mRNA. In some embodiments, the mRNA comprises an endogenously produced mRNA from a cell. In some embodiments, the mRNA comprises a mRNA produced from a heterologous gene of the cell. A mRNA is endogenously produced when it is produced from a gene of the cell that is not altered by genetic engineering. A heterologous gene refers to a gene that is transferred into the cell. The heterologous gene may be an additional copy of a gene that is already in the genome, may be maintained outside the genomic DNA, or may be integrated into the genome of the cell. In some embodiments, the cell comprises a prokaryotic or eukaryotic cell.

In some embodiments, the ribosome and/or translation factor binding site comprises a cap-independent binding site. A cap-independent binding site refers to a nucleic acid sequence that can recruit ribosomes and/or other translation factors in the presence or absence of a cap. In some embodiments, the translational activating region comprises an internal ribosomal entry site (IRES) or a ribosome and/or translation factor binding fragment thereof In some embodiments, the IRES comprises a Group 2 IRES or a ribosome and/or translation factor binding fragment thereof. In some embodiments, the IRES or IRES fragment comprises the IIIabc domain. In some embodiments, the IRES comprises a Group 4 IRES or a ribosome and/or translation factor binding fragment thereof. In some embodiments, the IRES or IRES fragment comprises the J-K region. In some embodiments, the IRES comprises a Group 1 IRES or a ribosome and/or translation factor binding fragment thereof. In some embodiments, the IRES comprises a Group 3 IRES or a ribosome and/or translation factor binding fragment thereof. IRES are known in the art and also further described herein. In some embodiments, the IRES comprises a HCV-like IRES structure. In some embodiments, the IRES comprises a EMCV-like IRES structure. In some embodiments, the ribosome and/or translation factor binding site is from or is derived from a viral, mammalian, or plant ribosomal binding site. In some embodiments, the translational activating region comprises an IRES from PTV-1, HCV, EMCV, CrPV, or fragments thereof, such as ribosome and/or translation factor binding fragments thereof. In some embodiments, the translational activating region comprises an IRES or IRES fragment from at least one of PTV-1, HCV, EMCV, and CrPV. In some embodiments, the translational activating region comprises an IRES or IRES fragment from at least two of PTV-1, HCV, EMCV, and CrPV. In some embodiments, the translational activating region comprises an IRES from PTV-1. In some embodiments, the ribosome binding site comprises an IRES from HCV. In some embodiments, the translational activating region comprises an IRES from EMCV. In some embodiments, the translational activating region comprises an IRES from CrPV. In some embodiments, the nucleic acid comprises 2 translational activating region. In some embodiments, the nucleic acid comprises at least, at most, or exactly 1, 2, 3, 4, 5, 6, 7, 8, 9, or 10 translational activating regions (or any range derivable therein). In some embodiments, the ribosome binding site comprises a nucleic acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NO:1-4 or 23-38. In some embodiments, the translation activating region comprises or consists of a ribosome binding site. In some embodiments, the translation activating region comprises or consists of a translation factor binding site. In some embodiments, the translation activating region excludes a ribosome binding site. In some embodiments, the translation activating region excludes a translation factor binding site. In some embodiments, the translation factor comprises eIF3 or eIF4G. Translation factors that are included or excluded in the disclosure include, for example, elFs, which include eIF1, eIF1A, eIF2, eIF3, eIF4, eIF4F, eIF4A, eIF4E, eIF4G, eIF5, eIF5A, eIF5B, and eIF6. In some embodiments, the translation factor comprises an IRES trans-activating factor (ITAF). The ITAF may comprise one or more of Annexin A2, CUGBP1, DAPS, FBP3, FUS, GRSF1, H-ferritin, HDMX, hnRNPA1, hnRNPC, hnRNPD, hnRNPE, hnRNPH2, hnRNPK, hnRNPL, hnRNPM, hnRNPQ, hnRNPR, HuR, La auto antigen, Mdm2, NF45, nPTB, nucleolin, p54nrb, PDCD4, PSF, PTB, RHA, SMAR, YB1, 4E-BP1, APP (AICD), eeF1A2, eIF3, eIF4A, eIF4GI, eIF5B, eL38, eS19, eS25, Gemin5, Hepsin, PINK1, Rackl, TCP80, uL1, uL24, uL5, VASH1, and TRMP.

In some embodiments, the nucleic acid comprises a modified nucleic acid. In some embodiments, the modification comprises at least one locked nucleic acid residue. In some embodiments, the modification comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, or 500 locked nucleic acid residues (or any derivable range therein).

In some embodiments, the modification comprises at least one phosphorothioate linkage. In some embodiments, the modification comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, or 500 phosphorothioate linkages (or any derivable range therein).

In some embodiments, the modification comprises an ethylene bridged nucleotide. In some embodiments, the modification comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, or 500 ethylene bridged nucleotides (or any derivable range therein).

In some embodiments, the modification comprises a peptide nucleic acid. In some embodiments, the modification comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, or 500 peptide nucleic acids (or any derivable range therein).

In some embodiments, the modification comprises a phosphorodiamidate morpholino. In some embodiments, the modification comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, or 500 phosphorodiamidate morpholino modified nucleic acids (or any derivable range therein).

In some embodiments, the modification comprises a 5′-vinyl-phosphonate. Also contemplated are nucleic acids with combinations of the modifications described herein.

In some embodiments, the targeting region comprises at least 12 nucleotides. In some embodiments, the targeting region is 20-50 nucleotides. In some embodiments, the targeting region is 30-50 nucleotides. In some embodiments, the targeting region is 35-45 nucleotides. In some embodiments, the targeting region is 40 nucleotides. In some embodiments, the targeting region is, is at least, is at most, is about, or is exactly 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nucleotides (or any derivable range therein).

In some embodiments, the nucleic acid is single stranded. In some embodiments, the nucleic acid is double stranded. In some embodiments, the nucleic acid does not further comprise a gene coding region. In some embodiments, the chimeric nucleic acid does not further comprise a gene coding region upstream of the translational activating region. In some embodiments, the chimeric nucleic acid does not further comprise a gene coding region downstream of the translational activating region. In some embodiments, the targeting region comprises an antisense nucleic acid. In some embodiments, the targeting region comprises a single stranded antisense nucleic acid. In some embodiments, the targeting region comprises a single stranded antisense RNA. In some embodiments, the nucleic acid does not comprise sense nucleic acid. The term “sense” of a nucleic acid molecule, particularly of a strand of DNA or RNA, refers to the nature of the roles of the strand and its complement in specifying a sequence of amino acids. Depending on the context, sense may have slightly different meanings. For example, DNA is sense if an RNA version of the same sequence is translated or translatable into protein, antisense if not.

In some embodiments, the nucleotide comprises a translational activating region that is 5′ of the targeting region. In some embodiments, the nucleic acid comprises a translational activating region that is 3′ of the targeting region. In some embodiments, the targeting region is complementary to at least a portion of a 3′UTR region of the mRNA. In some embodiments, the targeting region is complementary to at least a portion of a 5′UTR region of the mRNA. In some embodiments, the targeting region comprises at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 contiguous nucleic acids (or any range therein) that are complementary to a mRNA with at least, at most, or exactly 0, 1, 2, 3, 4, 5, 6, 7, 8, 9 or 10 mismatches in the complementary region. In some embodiments, the targeting region is complementary to at least a portion of the coding region of the mRNA. In some embodiments, the nucleic acid comprises deoxyribonucleic acid (DNA). In some embodiments, the nucleic acid is ribonucleic acid (RNA). In some embodiments, the targeting region comprises a region that is complementary to a tumor suppressor mRNA. In some embodiments, the tumor suppressor comprises PTEN. In some embodiments, the tumor suppressor comprises APC, IL2, TNFAIP3, ARHGEF12, JAK2, TP53, ATM, MAP2K4, TSC1, BCL11B, MDM4, TSC2, BLM, MEN1, VHL, BMPR1A, MLH1, WRN, BRCA1, MSH2, WT1, BRCA2, NF1, CARS, NF2, CBFA2T3, NOTCH1, CDH1, NPM1, CDH11, NR4A3, CDK6, NUP98, CDKN2C, PALB2, CEBPA, PML, CHEK2, PTEN, CREB1, RB1, CREBBP, RUNX1, CPLD, SDHB, DDXS, SDHD, EXT1, SMARCA4, EXT2, SMARCB1, FBXW7, SOCS1, FH, STK11, FLT3, SUFU, FOXP1, SUZ12, GPC3, SYK, IDH1, or TCF3.

In some embodiments, the targeting region comprises a region that is complementary to a mRNA from the SYNGAP1, ATP1A3, SCN1A, SCN2, or SIM1 gene. In some embodiments the targeting region comprises a region that is complementary to a mRNA from the SYNGAP1 gene. In some embodiments, the nucleic acid is used in a method for treating SYNGAP1-related intellectual disability or autism spectrum disorder. In some embodiments the targeting region comprises a region that is complementary to a mRNA from the ATP1A3 gene. In some embodiments, the nucleic acid is used in a method for treating ATP1A3-related neurological disorders, neurological disorders, alternating hemiplegia of childhood, rapid-onset dystonia parkinsonism, dystonia 12, cerebellar ataxia, areflexia, pes cavus, optic atrophy, or sensorineural hearing loss. In some embodiments the targeting region comprises a region that is complementary to a mRNA from the SCN1A gene. In some embodiments, the nucleic acid is used in a method for treating epileptic encephalopathy, epilepsy, epilepsy with febrile seizures, familial hemiplegic migraine, or Lennox-Gastaut syndrome. In some embodiments the targeting region comprises a region that is complementary to a mRNA from the SCN2 gene. In some embodiments, the nucleic acid is used in a method for treating neutropenia, severe congenital neutropenia, autosomal dominant neutropenia, nonimmune chronic idiopathic neutropenia, myeloid leukemia, AML, myelodysplastic syndrome, or myeloproliferative disease. In some embodiments the targeting region comprises a region that is complementary to a mRNA from the SIM1 gene. In some embodiments, the nucleic acid is used in a method for treating obesity, obesity due to SIM1 deficiency, and SIM1-related Prader-Willi-Like Syndrome.

The targeting region may target a mammalian RNA. In some embodiments, the targeting region target a human RNA. In some embodiments, the targeting region targets a viral RNA. In some embodiments, the targeting region targets a bacteria RNA. In some embodiments, the targeting region targets an eukaryotic RNA.

In some embodiments of the disclosure, the host cell is a bacterial cell. In some embodiments, the host cell is a mammalian cell. In some embodiments, the host cell is a human cell.

In some embodiments, the subject is one that has one allele of a target gene that encodes a wild type or functional protein and one variant allele of the target gene. In some embodiments, the variant allele of the target gene comprises a complete or partial loss of function mutation. In some embodiments, the target region is complementary to the mRNA transcribed from the wild type or functional allele of the gene. In some embodiments, the target mRNA encodes for Peptidylprolyl Isomerase B (PPIB) or wherein the target gene comprises PPIB. In some embodiments, the target mRNA encodes for a cell cycle inhibitor or wherein the target gene comprises a cell cycle inhibitor gene. In some embodiments, the haploinsufficiency disorder comprises Wolfram syndrome. In some embodiments, the target gene comprises Wolfram syndrome 1 (WFS1). In some embodiments, the haploinsufficiency disorder comprises Alzheimer's Disease. In some embodiments, the target gene comprises ATP binding cassette subfamily A member 7 (ABCA7). In some embodiments, the haploinsufficiency disorder comprises cancer, 1q21.1 deletion syndrome, 5q-syndrome in myelodysplastic syndrome (MDS), 22q11.2 deletion syndrome, CHARGE syndrome, cleidocranial dysostosis, Ehlers—Danlos syndrome, frontotemporal dementia caused by mutations in progranulin, GLUT1 deficiency (DeVivo syndrome), haploinsufficiency of A20, holoprosencephaly caused by haploinsufficiency in the Sonic Hedgehog gene, Holt—Oram syndrome, Marfan syndrome, Phelan—McDermid syndrome, polydactyly, or Dravet Syndrome.

Method embodiments of the disclosure include the treatment of cancer. In some embodiments, the cancer comprises breast cancer. In some embodiments, the breast cancer comprises triple negative breast cancer (TNBC). Methods may also include administration of additional therapeutics. In some embodiments, the method further comprises administration of a PI3K/mTOR inhibitor. In some embodiments, the inhibitor comprises BEZ235. In some embodiments, the subject is or has been determined to have a cancer that is resistant to PI3K/mTOR inhibition.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Illustration for antisense translation activating RNA. The elements showed for mRNA includes 5′ m7G cap, start codon (AUG) and poly (A) tail (An). When gRNA-IRES is added, it recruits more ribosomes (gray) to accelerate mRNA translation.

FIG. 2A-D. Daul-Luciferase reporter assay of different gRNA-IRES construction. The normalized luciferase amount is shown for different gRNAs with (A) PTV-1 IRES, (B) HCV IRES, (C) CrPV IRES and (D) EMCV IRES. Control is the ones fused with non-targeting guide RNA (white columns) and other guide RNAs target to 5′ UTR (gray), coding sequence (CDS,) and 3′ UTR. Statistical analyses performed with a two-tailed Student's t-test with unequal variance. Data expressed as mean±s.e.m. and normalized to signal of NT-IRES.

FIG. 3A-C gRNA-IRES system could increase endogenous protein level and affect cell function. (A) Western blots to analyze protein levels from HEK293T cells treated with either NT or gRNA which targets to PPIB mRNA (g2 to 5′ UTR, g3 to CDS, g4 and g5 to 3′ UTR) fused with PTV-1 IRES. Here, g4 most efficiently raised PPIB protein level. GAPDH is the loading control. (B) Western blots to analyze protein levels from HEK293T cells treated with either NT or gRNA targeting to 3′ UTR of p21^{CIP1/WAF1} (g1 and g2). (C) MTS assay to access HEK293T cell proliferation. Statistical analyses performed with a two-tailed Student's t-test with unequal variance. Data expressed as mean±s.e.m. and normalized to NT-PTV.

FIG. 4A-B gRNA-IRES system could raise disease-related protein level. (A) Western blots to analyze protein levels from HEK293T cells treated with either NT or gRNA which targets to WFS1 3′ UTR fused with PTV-1 IRES. GAPDH is the loading control. (B) Western blots to analyze protein levels from HEK293T cells treated with either NT or gRNA targeting to 3′ UTR of ABCA7. The α-tubulin is the loading control.

FIG. 5A-E. Design and engineering of taRNA (translation activating RNA). A. Schematic overview of the design and concept of taRNA. The bi-functional RNA molecule, taRNA, is made of gRNA domain and initiation machinery recruiting domain. On the translating mRNA, extra initiation factors and/or small ribosome would be recruited by taRNA to get close to start codon, thus increasing translation initiation rate. B. Vectors used in dual-luciferase assay (DLA). The target Firefly luciferase (Fluc) is encoded after ATTG Kozak sequence (−4 to −1 position) on the same vector with Renilla luciferase (Rluc). The taRNA (gRNA-IRES) is expressed after hU6 promotor on a separate vector. C. Dual-luciferase assay was used to screen different IRES with Fluc targeting gRNA, g5. Each IRES is named by their virus' name, and vector is the empty vector negative control (white). N=4 biological replicates. D. Screening guide RNAs targeting different positions of Fluc transcript, including targeting 5′ UTR (2^{nd} bar), CDS (bars 3-7) and 3′ UTR (bars 8-9). All are fused with PTV-1 IRES (PTV). N=3 biological replicates. E. Screening different lengths of g5 guide RNA (shown as SEQ ID NOS:36-40— top to bottom) fused with PTV-1 IRES (top 5 grey bars), using PTV-1 IRES without gRNA (0 nt) as negative control (bottom white bar). The gRNA sequences of different lengths are shown. All bar-graph values are shown as mean±SEM with data points. Student's t test: *p<0.05, **p<0.01.

FIG. 6A-H. Design and engineering of taRNA (translation activating RNA). A. Table summarizing the category group, length and initiation factors recruited of different IRES. B. Testing dual-luciferase reporters containing different Kozak sequences (position −4 to −1). The strength of ATTG, ATCC and CGCG gradiently increase and CACC is the canonical Kozak sequence. The g5-PTV taRNA was used to compare with empty vector control on different reporters. N=4 biological replicates. C. Comparing different non-targeting controls and IRES-only controls (grey) with empty vector control (white). N=4 biological replicates. D. RNA levels of Fluc kept similar between empty vector control (first bar) and taRNAs made from different IRES (bars 2-4), measured by RT-qPCR. N=3 biological replicates. E. More IRES from group 3 (FMDV), group 4 (PV) and human endogenous c-myc were utilized in taRNA constructions (bars 2-4), and tested for Fluc activating in dual-luciferase assay compared to empty vector control (first bar). F. Repeating taRNA screening with different IRES in SK-MEL-28 cells and G. in MDA-MB-231 cells. H. Two possible orientations were tested together with PTV-1 IRES alone as control. All bar-graph values are shown as mean±SEM with data points. Student's t test: *P<0.05, **P<0.01, ***P<0.001, ****P<0.0001.

FIG. 7A-E. Validation of taRNA for increasing endogenous gene expression. A. Two guide RNAs targeting 3′ UTR of human PPIB mRNA, g4 and g5, individually fused with PTV-1 IRES were tested in HEK293T cells. Representative immunoblotting images analyzing PPIB protein level were shown and GAPDH was used as loading control. PPIB protein level was upregulated about 1.4-fold compared to non-targeting control by quantifying immunoblots. N=3 biological replicates. B. Two guide RNAs targeting 3′ UTR of human p21^{CIP1/WAF1} mRNA were used in taRNAs. Similar immunoblotting was performed as A, and guide RNA g2 was effective but not g1. Quantification was performed with 4 biological replicates. C. Two guide RNAs targeting 3′ UTR of human PTEN mRNA were used in taRNAs. PTEN-g2-PTV taRNA increased PTEN protein level around 1.7-fold compared to non-targeting control. N=4 biological replicates. D. Quantification of immunoblotting data with PTEN-g2-PTV taRNA treating MDA-MB-231 cells compared to non-targeting control. E. MDA-MB-231 cells (stably expressing Fluc) transfected with either PTEN-activating taRNA (PTEN-g2-PTV) or non-targeting control (NT-PTV) were treated with gradient doses of BEZ235. Cell viability was measured by Fluc signal. All bar-graph values are shown as mean±SEM with data points. Student's t test: *P<0.05, **P<0.01.

FIG. 8A-D. Validation of taRNA for increasing endogenous gene expression. A. The on-target taRNA (gray) causes no significant change on targeted PPIB mRNA amount compared to non-targeting taRNA (white) by RT-qPCR measurement. N=3 biological replicates B. No significant changes at RNA level with p21 targeting taRNA and with PTEN targeting taRNA in C. N=3 biological replicates in both B and C.D. Immunoblotting images revealed that PTEN targeting PTV increased PTEN expression in MDA-MB-231 cells. All bar-graph values are shown as mean±SEM with data points.

FIG. 9A-D. Truncated IRES domains for initiation factor recruitment are effective in taRNAs. A. Truncated HCV IRES were fused with Fluc targeting g5 to be tested in dual-luciferase assay for activating Fluc expression. HCV-ΔII indicates the HCV IRES without domain II and HCV-IIIabc means the IIIa-Mb-IIIc region of HCV IRES. N=4 biological replicates. B. Immunoblots measuring PTEN targeting taRNA effects with HCV-IIIabc in HEK293T cells to increase PTEN expression. GAPDH was the loading control. Quantification was done using 6 biological replicates. C. Dual-luciferase assay shows different regions from CSFV, HCV and PTV-1 IRES (bars 3-6) increased target Fluc expression. The HCV-U228C (bar 2) is HCV-IIIabc sequence with single U228C mutation. N=4 biological replicates. D. Dual-luciferase assay shows J-K region of EMCV (EMCV-JK) is effective as taRNA to increase Fluc expression compared to empty vector control, and slightly better than EMCV full length. N=4 biological replicates. All bar-graph values are shown as mean±SEM with data points. Student's t test: *P<0.05, **P<0.01, ****P<0.0001.

FIG. 10A-D. Truncated IRES domains for initiation factor recruitment are effective in taRNAs. A. Summary of domain lengths and functions of different IRES. B. The HCV-ΔII-ΔIIIb regions failed to upregulate target Fluc expression compared to empty vector and non-targeting control (NT-ΔII-ΔIIIb) in dual-luciferase assay. N=4 biological replicates. C. Fluc targeting g5-HCV-IIIabc (g5-HCV-IIIabc) increase Fluc protein level about 1.4-fold compared to non-targeting control (NT-HCV-IIIabc, white). N=4 biological replicates. D. Immunoblotting images analyzing PTEN level in HEK293T cells with PTEN-targeting HCV-IIIabc taRNA treatment, compared to non-targeting control (NT). GAPDH is the loading control. All bar-graph values are shown as mean±SEM with data points. Student's t test: **P <0.01.

### DETAILED DESCRIPTION OF THE INVENTION

RNA molecule regulation has emerged as an important therapeutics target and tool to control gene expression. RNA therapies enable one to target traditional “undruggable” targets without permanently altering the genome, and its programmability make treatment cost-effective and easy to combine with other drugs. To broaden the ability to efficiently increase targeted protein levels at the translational level, the inventors developed an antisense-translation-activating RNA technology. It was found that delivering effector RNAs to a target transcript boosts translation from the RNA and thereby increases the amount of protein produced.

In order to develop this approach, the inventors deployed internal ribosome entry site (IRES) RNAs as a ribosome-recruiting element. IRES elements have been studied for two decades as a cis-element to recruit the 40S ribosomal subunit through cap-independent mechanisms. Recent structural studies revealed that the IRES bound ribosomes could still bind and translate another mRNA in a cap-dependent manner, which inspired the inventors to test whether adding a guide-RNA on an IRES would cause the IRES-captured ribosomes to accumulate near targeted mRNA and thereby accelerate translation. Based on this hypothesis, the inventors designed the gRNA-IRES single RNA molecule as a tool to boost specific protein levels.

### I. Ribosome Binding Sites and Nucleic Acid Sequence Embodiments

Described below are exemplary embodiments that are useful in the nucleic acids of the disclosure.

A. Internal Ribosome Binding Sites

PTV-1 IRES sequence:
(SEQ ID NO: 1)
UACUUGGUUAUGAAUUCAUUGUAUUAACCCCUCUGAAAGACCUGCUCUGG
CGCGAGCUAAAGCGCAAUUGUCACCAGGUAUUGCACCAAUGGUGGCGACA
GGGUACAGAAGAGCAAGUACUCCUGACUGGGUAAUGGGACUGCAUUGCAU
AUCCCUAGGCACCUAUUGAGAUUUCUCUGGGGCCCACCAGCGUGGAGUUC
CUGUAUGGGAAUGCAGGACUGGACUUGUGCUGCCUGACAGGGUCGCGGCU
GGCCGUCUGUACUUUGUAUAGUCAGUUGAAACUCACC.
HCV IRES sequence:
(SEQ ID NO: 2)
TCCCCTGTGAGGAACTACTGTCTTCACGCAGAAAGCGTCTAGCCATGGCG
TTAGTATGAGTGTCGTGCAGCCTCCAGGCCCCCCCCTCCCGGGAGAGCCA
TAGTGGTCTGCGGAACCGGTGAGTACACCGGAATTGCCAGGACGACCGGG
TCCTTTCTTGGATCAATCCCGCTCAATGCCTGGAGATTTGGGCGTGCCCC
CGCGAGACTGCTAGCCGAGTAGTGTTGGGTCGCGAAAGGCCTTGTGGTAC
TGCCTGATAGGGTGCTTGCGAGTGCCCCGGGAGGTCTCGTAGACCGTGCA
CC.
EMCV IRES sequence:
(SEQ ID NO: 3)
GAGGGCCCGGAAACCTGGCCCTGTCTTCTTGACGAGCATTCCTAGGGGTC
CTTTCCCTCTCGCCAAAGGAATGCAAGGTCTGTTGAATGTCGTGAAGGAA
GCAGTTCCTCTGGAAGCTTCTTGAAGACAAACAACGTCTGTAGCGACCCT
TTGCAGGCAGCGGAACCCCCCACCTGGCGACAGGTGCCTCTGCGGCCAAA
AGCCACGTGTATAAGATACACCTGCAAAGGCGGCACAACCCCAGTGCCAC
GTTGTGAGTTGGATAGTTGTGGAAAGAGTCAAATGGCTCTCCTCAAGCGT
ATTCAACAAGGGGCTGAAGGATGCCCAGAAGGTACCCCATTGTATGGGAT
CTGATCTGGGGCCTCGGTGCACATGCTTTACATGTGTTTAGTCGAGGTTA
AAAAAACGTCTAGGCCCCCCGAACCACGGGGACGTGGTTTTCCTTTGAAA
AACACGATGATAA.
CrPV IRES sequence:
(SEQ ID NO: 4)
AGCAAAAATGTGATCTTGCTTGTAAATACAATTTTGAGAGGTTAATAAAT
TACAAGTAGTGCTATTTTTGTATTTAGGTTAGCTATTTAGCTTTACGTTC
CAGGATGCCTAGTGGCAGCCCCACAATATCCAGGAAGCCCTCTCTGCGGT
TTTTCAGATTAGGTAGTCGAAAAACCTAAGAAATTTACCTGCTACATTTC
AAGA.
The IRES sequences (upper case) may be added to
targeting region (lower case underline) and linker
(upper case italic), for example, NT-PTV-1 IRES:
(SEQ ID NO: 5)
tgacagcccacatggcattccacttatcactggcatccttTTATTUACUU
GGUUAUGAAUUCAUUGUAUUAACCCCUCUGAAAGACCUGCUCUGGCGCGA
GCUAAAGCGCAAUUGUCACCAGGUAUUGCACCAAUGGUGGCGACAGGGUA
CAGAAGAGCAAGUACUCCUGACUGGGUAAUGGGACUGCAUUGCAUAUCCC
UAGGCACCUAUUGAGAUUUCUCUGGGGCCCACCAGCGUGGAGUUCCUGUA
UGGGAAUGCAGGACUGGACUUGUGCUGCCUGACAGGGUCGCGGCUGGCCG
UCUGUACUUUGUAUAGUCAGUUGAAACUCACC.

Further IRE S embodiments include:

PV-IRES:
(SEQ ID NO: 41)
GACGCACAAAACCAAGTTCAATAGAAGGGGGTACAAACCAGTACCACCAC
GAACAAGCACTTCTGTTTCCCCGGTGATGTCGTATAGACTGCTTGCGTGG
TTGAAAGCGACGGATCCGTTATCCGCTTATGTACTTCGAGAAGCCCAGTA
CCACCTCGGAATCTTCGATGCGTTGGTTAGCACTCAACCCCAGAGTGTAG
CTTAGGCTGATGAGTCTGGACATCCCTCACCGGTGACGGTGTTCCAGGCT
GCGTTGGCGGCCTACCTATGGCTAACGCATGGGACGCTAGTTGTGAACAA
GGTGTGAAGAGCCTATTGAGCTACATAAGAATCCTCCGGCCCCTGAATGC
GGCTAATCCCAACCTCGGAGCAGGTGGTTCACAAACCAGTGATTGGCCTG
TCGTAACGCGCAAGTCCGTGGCGGAACCGACTACTTTGGGTGTCCGTGTT
TCCTTTTATTTTATTGTGGCTGCTTATGGTGACAATCACAGATTGTTATC
ATAAAGCGAATTGGATTGGCCATCCGGTGAAAGTGAGACTCATTATCTAT
CTGTTTGCTGGATCCGCTCCATTGAGTGTGTTTACTCTAAGTACAATTTC
AACAGTTATTTCAATCAGACAATTGTATCATAATG;
FMDV-IRES:
(SEQ ID NO: 42)
CACGATTTAAGCAGGTTTCCACAACTGATAAAACTCGTGCAACTTGAAAC
TCCGCCTGGTCTTTCCAGGTCTAGAGGGGTTACACTTTGTACTGTGCTCG
ACTCCACGCCCGGTCCACTGGCGGGTGTTAGTAGCAGCACTGTTGTTTCG
TAGCGGAGCATGGTGGCCGTGGGAACTCCTCCTTGGTGACAAGGGCCCAC
GGGGCCGAAAGCCACGTCCAGACGGACCCACCATGTGTGCAACCCCAGCA
CGGCAACTTTTACTGCGAACACCACCTTAAGGTGACACTGGTACTGGTAC
TCGGTCACTGGTGACAGGCTAAGGATGCCCTTCAGGTACCCCGAGGTAAC
ACGGGACACTCGGGATCTGAGAAGGGGATTGGGACTTCTTTAAAAGTGCC
CAGTTTAAAAAGCTTCTACGCCTGAATAGGCGACCGGAGGCCGGCGCCTT
TCCATTAC;
and
FMDV-IRES
(SEQ ID NO: 43)
CAACCCTTGCCGCATCCACGAAACTTTGCCCATAGCAGCGGGCGGGCACT
TTGCACTGGAACTTACAACACCCGAGCAAGGACGCGACTCTCCCGACGCG
GGGAGGCTATTCTGCCCATTTGGGGACACTTCCCCGCCGCTGCCAGGACC
CGCTTCTCTGAAAGGCTCTCCTTGCAGCTGCTTAGACGCTGGATTTTTTT
CGGGTAGTGGAAAACCAGCAGCCTCCCGCGACGATGCCCCTCAACGTTAG
CTTCACCAACAGGAACTATGACCTCGACTACGACTCGGTGCAGCCGTATT
TCTACTGCGACGAGGAGGAGAACTTCTACCAGCAGCAGCAGCAGAGCGAG
CT.

Other exemplary IRES molecules are included in the table below:

| | | SEQ |
|---|---|---|
| | | ID |
| IRES | Sequence | NO: |
| ABPV_ | CACAACATGGTTACCCATAGATTGAGGAAATTTC | 23 |
| IGRpred | CAATAAACTCAGTATTAAGGCTTGTTGTGTTGGA | |
| | CAAGGTGCCCTATTTAGGGTGAGGAGCCTTACTG | |
| | GCAGCCCCAGTGAATCCTCCATTGGATAGGAACA | |
| | GCTATATTGGGTAGTTGTAGCAGTTGTATTCAAA | |
| | TGAATGCAGCGTTCCGAAATATCATACCT | |
| AEV | TTTGAAAGAGGCCTCCGGAGTGTCCGGAGGCTCT | 24 |
| | CTTTCGACCCAACCCATACTGGGGGGTGTGTGGG | |
| | ACCGTACCTGGAGTGCACGGTATATATGCATTCC | |
| | CGCATGGCAAGGGCGTGCTACCTTGCCCCTTGAC | |
| | GCATGGTATGCGTCATCATTTGCCTTGGTTAAGC | |
| | CCCATAGAAACGAGGCGTCACGTGCCGAAAATC | |
| | CCTTTGCGTTTCACAGAACCATCCTAACCATGGG | |
| | TGTAGTATGGGAATCGTGTATGGGGATGATTAGG | |
| | ATCTCTCGTAGAGGGATAGGTGTGCCATTCAAAT | |
| | CCAGGGAGTACTCTGGCTCTGACATTGGGACATT | |
| | TGATGTAACCGGACCTGGTTCAGTATCCGGGTTG | |
| | TCCTGTATTGTTACGGTGTATCCGTCTTGGCACAC | |
| | TGAAAGGGTATTTTTGGGTAATCCTTTCCTACTGC | |
| | CTGATAGGGTGGCGTGCCCGGCCACGAGAGATTA | |
| | AGGGTAGCAATTTAAAC | |
| ALPV_ | AATTACTAATTTGATCTTTAGGTTATAATGTTAGG | 25 |
| IGRpred | ACTATAAAAATTAGCTTAATGCATTTAGTAATTT | |
| | AAGGCTTAGTTATTTAACTTTACTTATCAAGATG | |
| | GCCGTTGGCAGCCCCACGAAATCTAGATTAGTCC | |
| | GAATGTCCTATTTTGATTAGGTGGTCAGATAGGT | |
| | CAGAAACTCACCT | |
| BQCV_ | CCAACAATGTGATCTTGCTTGCGGAGGCAAAATT | 26 |
| IGRpred | TGCACAGTATAAAATCTGCAAGTAGTGCTATTGT | |
| | TGGAATCACCGTACCTATTTAGGTTTACGCTCCA | |
| | AGATCGGTGGATAGCAGCCCTATCAATATCTAGG | |
| | AGAACTGTGCTATGTTTAGAAGATTAGGTAGTCT | |
| | CTAAACAGAACAATTTACCT | |
| BVDV1 | GTATACGAGAATTAGAAAAGGCACTCGTATACGT | 27 |
| | ATTGGGCAATTAAAAATAATAATTAGGCCTAGGG | |
| | AACAAATCCCTCTCAGCGAAGGCCGAAAAGAGG | |
| | CTAGCCATGCCCTTAGTAGGACTAGCATAATGAG | |
| | GGGGGTAGCAACAGTGGTGAGTTCGTTGGATGGC | |
| | TTAAGCCCTGAGTACAGGGTAGTCGTCAGTGGTT | |
| | CGACGCCTTGGAATAAAGGTCTCGAGATGCCACG | |
| | TGGACGAGGGCATGCCCAAAGCACATCTTAACCT | |
| | GAGCGGGGGTCGCCCAGGTAAAAGCAGTTTTAA | |
| | CCGACTGTTACGAATACAGCCTGATAGGGTGCTG | |
| | CAGAGGCCCACTGTATTGCTACTAAAAATCTCTG | |
| | CTGTACATGGCACATGGAGTTGATCACAAATGAA | |
| | CTTTTATACAAAACATACAAACAAAAACCCGTCG | |
| | GGGTGGAGGAACCTGTTTATGATCAGGCAGGTGA | |
| | TCCCTTATTTGGTGAAAGGGGAGCAGTCCACCCT | |
| | CAATCGACGCTAAAGCTCCCACACAAGAGAGGG | |
| | GAACGCGATGTTCCAACCAACTTGGCATCCTTAC | |
| | CAAAAAGAGGTGACTGCAGGTCGGGTAATAGCA | |
| | GAGGACCTGTGAGCGGGATCTACCTGAAGCCAG | |
| | GGCCACTATTTTACCAGGACTATAAAGGTCCCGT | |
| | CTATCACAGGGCCCCGCTGGAGCTCTTTGAGGAG | |
| | GGATCCATGTGTGAAACGACTAAACGGATAGGG | |
| | AGAGTAACTGGAAGTGACGGAAAGCTGTACCAC | |
| | ATTTATGTGTGTATAGATGGATGTATAATAATAA | |
| | AAAGTGCCACGAGAAGTTACCAAAGGGTGTTCA | |
| | GGTGGGTCCATAATAGGCTTGACTGCCCTCTATG | |
| | GGTCACAACTTGCTCAGACACGAA | |
| crTMV_ | GAATTCGTCGATTCGGTTGCAGCATTTAAAGCGG | 28 |
| IREScp | TTGACAACTTTAAAAGAAGGAAAAAGAAGGTTG | |
| | AAGAAAAGGGTGTAGTAAGTAAGTATAAGTACA | |
| | GACCGGAGAAGTACGCCGGTCCTGATTCGTTTAA | |
| | TTTGAAAGAAGAAA | |
| CSFV + 3 | GcATACGAGGTTAGTTCATTCTCGTATACACGATT | 29 |
| | GGACAAATCAAAATTATAATTTGGTTCAGGGCCT | |
| | CCCTCCAGCGACGGCCGAACTGGGCTAGCCATGC | |
| | CCATAGTAGGACTAGCAAAACGGAGGGACTAGC | |
| | CATAGTGGCGAGCTCCCTGGGTGGTCTAAGTCCT | |
| | GAGTACAGGACAGTCGTCAGTAGTTCGACGTGAG | |
| | CAGAAGCCCACCTCGAGATGCTACGTGGACGAG | |
| | GGCATGCCAAGACACACCTTAACCCTAGCGGGG | |
| | GTCGCTAGGGTGAAATCACACCACGTGATGGGA | |
| | GTACGACCTGATAGGGCGCTGCAGAGGCCCACTA | |
| | TTAGGCTAGTATAAAAATCTCTGCTGTACATGGC | |
| | ACatG | |
| DCV_IGR | GTTAAGATGTGATCTTGCTTCCTTATACAATTTTG | 30 |
| | AGAGGTTAATAAGAAGGAAGTAGTGCTATCTTAA | |
| | TAATTAGGTTAACTATTTAGTTTTACTGTTCAGGA | |
| | TGCCTATTGGCAGCCCCATAATATCCAGGACACC | |
| | CTCTCTGCTTCTTATATGATTAGGTTGTCATTTAG | |
| | AATAAGAAAATAACCT | |
| TRV_5NTR | CAAAATTGCGTGCGAGAAAGCACGCAAATCAAA | 31 |
| | GTCTAGTGCGTAATTCACTCACTACCGGCGTAAT | |
| | TTGTGGTTATGCTATTGCGTTGAGAGTGTTGTGG | |
| | GCGTAGTACGGGGGCTTTTGGTTTGTGTGTGATT | |
| | AATATGCATTCCCAGTTTTGTTTAGTTTTAGATTA | |
| | CTGATTTATTTTTCGAACTACCCGAATTTATTTAG | |
| | GCTCTTCGAGAAATAATGATGAACTGTCCTCAAC | |
| | AAGTATGAAAAGCAATTATTTGTGAAGTTACTTG | |
| | TTGTTGTAAGATTATTGACCTCTTAGATTTTTCTA | |
| | AGTTGTAATGCTTTGTTTTCTGATTGACTAGATTA | |
| | TGAATCCAATTAAAAGGAGTAGTGGTCTAATATA | |
| | GTCTGTGTGACCTGCAGGCATTTTGTGAAAAGGG | |
| | TAAAGTATGAAAGCTACTCTCAGAAAAGTACTTA | |
| | TGTATTGATGAGAGCCTTAAAATGACTTTATATT | |
| | CACAAAACTGCTGGAAGACAATGATCTGGGGTAT | |
| | CACATTCCCTCTTAGGTTAAGTTTCGCACTACTAG | |
| | GAATTTTTGCGAAAATTCTTAATTCCATTCTTATG | |
| | GTGTAGTGTTTGTTCTTTTCAATTGGGGCGTATCT | |
| | CTCTTTCGCAGAGACCCTGGCCCAACCCTGAAAT | |
| | GAATAAAATTTTACAAAAATTCATCGAAAATCGA | |
| | CCTTCTAG | |
| ERBV_ | ATTGATGTGTTGGTCGTTTGCCAATCGGAGGGCG | 32 |
| 189-920 | ACAGGTCGTTTGCCAATCGGAGGGCGACAGGCA | |
| | CAGGTCGCTCCGAGTTCTAGTAGTGTGGGAACTT | |
| | GTTACTACTGATGAAACGAGGTAGTGACACTGAC | |
| | TACCTGCGAACGAGGTCGGGGCCCTCCCTTCTTC | |
| | CTTCACCCAACTTTCACTTTTCGTTCCACTTTAGC | |
| | AGGGGTCTTCTTTCTATCCCCCTGGCGGCATTGG | |
| | AACTAGCCGTCGCGTCTTAACGCGCAGCCCTGAA | |
| | GGCCCCACACCTTGTGGATCTTGCCGTGGGTATG | |
| | TTTCTGGCATGTGTTTCTCAAGCCTGCAACCGAA | |
| | GCCGAACAGCCACATGAACAGTTTGAGCGTGGTA | |
| | GCGCTGTGTGAGTTGGCGGTGGATCCCCCTCGTG | |
| | GTAACACGAGCCCCCGTGGCCAAAAGCCCAGTGT | |
| | TTACAGCACCTCTCACATCCAGGACGACCCCATC | |
| | CTGGCGCTCACTCTTAGTAGTATGGCTTAGTACG | |
| | CATTAGGTGGTAAGCCGAGCTCTCCCTCGGCCTT | |
| | GTTCTGAATGCACACATGTCTAGGGGCTAAGGAT | |
| | GTCCTACAGGTACCCGCACGTAACCTTCAGAGAG | |
| | TGCGGATCTGAGTAGGAGACCGTGGTGCACTGCT | |
| | TTACAGATGCAGCCCCGGTTTAAAAAGCGTCTAT | |
| | GCCCCTACAGGGTAGCGGTGGGCCGCGCCCTTTC | |
| | CTTTTAAAACTACTTGTTCTATGGTGACAATGGC | |
| | AGGAAACATGAT | |
| EMCV-R_ | CGGTGTGCGTTTGTCTATATGTTATTTTCCACCAT | 33 |
| 315-845 | ATTGCCGTCTTTTGGCAATGTGAGGGCCCGGAAA | |
| | CCTGGCCCTGTCTTCTTGACGAGCATTCCTAGGG | |
| | GTCTTTCCCCTCTCGCCAAAGGAATGCAAGGTCT | |
| | GTTGAATGTCGTGAAGGAAGCAGTTCCTCTGGAA | |
| | GCTTCTTGAAGACAAACAACGTCTGTAGCGACCC | |
| | TTTGCAGGCAGCGGAACCCCCCACCGGGCGACA | |
| | GGTGCCTCTGCGGCCAAAAGCCACGTGTATAAGA | |
| | TACACCTGCAAAGGCGGCACAACCCCAGTGCCAC | |
| | GTTGTGAGTTGGATAGTTGTGGAAAGAGTCAAAT | |
| | GGCTCTCCTCAAGCGTATTCAACAAGGGGCTGAA | |
| | GGATGCCCAGAAGGTACCCCATTGTATGGGATCT | |
| | GATCTGGGGCCTCGGTGCACATGCTTTACATGTG | |
| | TTTAGTCGAGGTTAAAAAGCGTCTAGGCCCCCCG | |
| | AACCACGGGGACGTGGTTTTCCTTTGAAAAACAC | |
| | GATGATAATATGGCCACAACC | |
| RhPV_ | GATAAAAGAACCTATAATCCCTTCGCACACCGCG | 34 |
| 5NCR | TCACACCGCGCTATATGCTGCTCATTAGGAATTA | |
| | CGGCTCCTTTTTTGTGGATACAATCTCTTGTATAC | |
| | GATATACTTATTGTTAATTTCATTGACCTTTACGC | |
| | AATCCTGCGTAAATGCTGGTATAGGGTGTACTTC | |
| | GGATTTCCGAGCCTATATTGGTTTTGAAAGGACC | |
| | TTTAAGTCCCTACTATACTACATTGTACTAGCGTA | |
| | GGCCACGTAGGCCCGTAAGATATTATAACTATTT | |
| | TATTATATTTTATTCACCCCCCACATTAATCCCAG | |
| | TTAAAGCTTTATAACTATAAGTAAGCCGTGCCGA | |
| | AACGTTAATCGGTCGCTAGTTGCGTAACAACTGT | |
| | TAGTTTAATTTTCCAAAATTTATTTTTCACAATTT | |
| | TTAGTTAAGATTTTAGCTTGCCTTAAGCAGTCTTT | |
| | ATATCTTCTGTATATTATTTTAAAGTTTATAGGAG | |
| | CAAAGTTCGCTTTACTCGCAATAGCTATTTTATTT | |
| | ATTTTAGGAATATTATCACCTCGTAATTATTTAAT | |
| | TATAACATTAGCTTTATCTATTTATA | |
| HAV | TCCCTCTTGGAAGTCCCTGGTGAGGGGACTTGAT | 35 |
| | ACCTCACCGCCGTTTGCCTAGGCTATAGGCTAAA | |
| | TTTTCCCTTTCCCTTTTCCCTTTCCCATTCCCTTTT | |
| | GCTTGTAAATATTGATTCCTGCAGGTTCAGGGTT | |
| | CTTAAATCTGTTTCTCTATAAGAACACTCATTTTC | |
| | ACGCTTTCTGTCTTCTTTCTTCCAGGGCTCTCCCC | |
| | TTGCCCTAGGCTCTGGCCGTTGCGCCCGGCGGGG | |
| | TCAACTCCATGATTAGCATGGAGCTGTAGGAGTC | |
| | TAAATTGGGGACACAGATGTTTGGAACGTCACCT | |
| | TGCAGTGTTAACTTGGCTTTCATGAATCTCTTTGA | |
| | TCTTCCACAAGGGGTAGGCTACGGGTGAAACCTC | |
| | TTAGGCTAATACTTCTATGAAGAGATGCCTTGGA | |
| | TAGGGTAACAGCGGCGGATATTGGTGAGTTGTTA | |
| | AGACAAAAACCATTCAACGCCGGAGGACTGACT | |
| | CTCATCCAGTGGATGCATTGAGTGGATTGACTGT | |
| | CAGGGCTGTCTTTAGGCTTAATTCCAGACCTCTCT | |
| | GTGCTTAGGGCAAACATCATTTGGCCTTAAATGG | |
| | GATTCTGTGAGAGGGGATCCCTCCATTGACAGCT | |
| | GGACTGTTCTTTGGGGCCTTATGTGGTGTTTGCCT | |
| | CTGAGGTACTCAGGGGCATTTAGGTTTTTCCTCAT | |
| | TCTTAAATAATAATGAACATGTCTAGACAAGGTA | |
| | TTTTCCAGACTGTTGGGAGTGGTCTTGACCACAT | |
| | CCTGTCTTTGGCAGACATTGAGGAAGAGCAAATG | |
| | ATTCAATCAGTTGATAGGACTGCAGTGACTGGTG | |
| | CTTCTTATTTTACTTCTGTGGATCAATCT | |

### B. Exemplary Targeting Region Embodiments

The oligonucleotides exemplify targeting region embodiments (also referred to as guide RNA or gRNA) to specific mRNAs.

Lux gRNA-1:
(SEQ ID NO: 6)
ggtggctttaccaacagtaccggattgccaagcttgggct:
Lux gRNA-2
(SEQ ID NO: 7)
cgctgggcccttcttaatgtttttggcatcttccatggtg;
Lux gRNA-3
(SEQ ID NO: 8)
atggcgctgggcccttcttaatgtttttggcatcttccat;
Lux gRNA-4
(SEQ ID NO: 9)
ggtagaatggcgctgggcccttcttaatgtttttggcatc;
Lux gRNA-5
(SEQ ID NO: 10)
caggtcgactctagactcgaggctagcgagctcgtttaaa;
Lux gRNA-7:
(SEQ ID NO: 11)
gctcagcggtggcagcagccaactcagcttcctttcgggc;
Lux gRNA-8
(SEQ ID NO: 12)
tcatgtctgctcgaagcggccgccccaaggggttatgcta;
PPIB-gRNA-2:
(SEQ ID NO: 13)
ccacaggcggaggcgaaagcagcccggacagctgaggccg;
PPIB-gRNA-3:
(SEQ ID NO: 14)
caaggagcaccttcatgttgcgttcggagaggcgcagcat;
PPIB-gRNA-4:
(SEQ ID NO: 15)
cctgcacagacggtcactcaaagaaagatgtccctgtgcc;
PPIB-gRNA-5:
(SEQ ID NO: 16)
gaatgtgaggggagtgggtccgctccaccagatgccagca;
p21-gRNA-1:
(SEQ ID NO: 17)
agagcgggcctttgaggccctcgcgcttccaggactgcag;
p21-gRNA-2:
(SEQ ID NO: 18)
ggggggcagggggcggccagggtatgtacatgaggaggtg;
WFS1-gRNA-4:
(SEQ ID NO: 19)
atggcaacatgcactggaagctcctcgtggcggaccatcc;
WFS1-gRNA-5:
(SEQ ID NO: 20)
ttgtcggggtccacgcaatctacacatggtcgcaaggtct;
ABCA7-gRNA-1:
(SEQ ID NO: 21)
attcccagggcctccccgcggccccgcaggggagggaggc.

### II. Oligonucleotides

The term “nucleoside” refers to a unit made up of a heterocyclic base and its sugar. The term “nucleotide” refers to a nucleoside having a phosphate group on its 3′ or 5′ sugar hydroxyl group. The term “oligonucleotide” or “nucleic acid” refers to a plurality of joined nucleotide units formed in a specific sequence from naturally occurring bases and pentofuranosyl groups joined through a sugar group by native phosphodiester bonds. This term refers to both naturally occurring and synthetic species formed from naturally occurring subunits.

The nucleic acids of the disclosure may be ribonucleic acids or deoxyribose nucleic acids. In some embodiments, the nucleic acids are modified. Modifications include altered sugar moieties, altered base moieties or altered inter-sugar linkages. The nucleic acids may be joined via either natural phosphodiester bonds or other linkages, including the four atom linkers. Although the linkage generally is from the 3′ carbon of one nucleoside to the 5′ carbon of a second nucleoside, the term nucleic acid can also include other linkages such as 2′-5′ linkages.

Nucleic acids also can include other modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true nucleic acid species. All such compounds are considered to be modified nucleic acids. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

In some embodiments, the nucleic acid comprises a modified nucleic acid. These modified nucleic acids may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleotide linkages are intended to replace naturally-occurring phosphodiester-5′-methylene linkages with four atom linking groups to confer nuclease resistance and enhanced cellular uptake to the resulting compound. In some embodiments, the backbone of the nucleic acid is modified to comprise a phosphorothioate. The phosphorothioate bond may substitute a sulfur atom for a non-bridging oxygen in the phosphate backbone of an oligonucleotide. This modification renders the internucleotide linkage resistant to nuclease degradation. Phosphorothioate bonds can be introduced between the last 3-5 nucleotides at the 5′- or 3′-end of the oligonucleotide to inhibit exonuclease degradation. Including phosphorothioate bonds throughout the entire oligonucleotide will help reduce attack by endonucleases as well.

Methods for the preparation of nucleic acids are disclosed. Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Nucleic acids linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TentaGel Support—an aminopolyethyleneglycol derivatized support or Poros —a copolymer of polystyrene/divinylbenzene. Attachment and cleavage of nucleotides and oligonucleotides can be affected via standard procedures [55]. As used herein, the term solid support further includes any linkers (e.g., long chain alkyl amines and succinyl residues) used to bind a growing oligonucleotide to a stationary phase such as CPG.

A. Locked Nucleotides

A locked nucleic acid (LNA or Ln), also referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. Such nucleic acids are synthesized chemically and are commercially available. The locked ribose conformation enhances base stacking and backbone pre-organization. This significantly increases the hybridization properties (melting temperature) of oligonucleotides.

B. Ethylene Bridged Nucleotides

Ethylene-bridged nucleic acids (ENA or En) are modified nucleotides with a 2′-O, 4′C ethylene linkage. Like locked nucleotides, these nucleotides also restrict the sugar puckering to the N-conformation of RNA.

C. Peptide Nucleic Acids

Peptide nucleic acids (PNA or Pn) mimic the behavior of DNA and binds complementary nucleic acid strands. The term, “peptide,” when used herein may also refer to a peptide nucleic acid. PNA is an artificially synthesized polymer similar to DNA or RNA. DNA and RNA have a deoxyribose and ribose sugar backbone, respectively, whereas PNA's backbone is composed of repeating N-(2-aminoethyl)-glycine units linked by peptide bonds. The various purine and pyrimidine bases are linked to the backbone by a methylene bridge (—CH2—) and a carbonyl group (—(C═O)—). PNAs are depicted like peptides, with the N-terminus at the first (left) position and the C-terminus at the last (right) position.

Since the backbone of PNAs contains no charged phosphate groups, the binding between PNA/DNA strands is stronger than between DNA/DNA strands due to the lack of electrostatic repulsion. PNAs are not easily recognized by either nucleases or proteases, making them resistant to degradation by enzymes. PNAs are also stable over a wide pH range. In some aspects, the PNAs described herein have improved cytosolic delivery over other PNAs.

D. Phosphorodiamidate Morpholino Oligonucleotides

Phosphorodiamidate morpholino oligomers (PMO or Po) are short single-stranded DNA analogs that are built upon a backbone of morpholine rings connected by phosphorodiamidate linkages. PMOs are uncharged nucleic acid analogs that are less likely to interact with proteins. PMOs bind to complementary sequences of target mRNAs by Watson-Crick base pairing and block mRNA translation through sequence-specific blockade. PMOs are resistant to nucleases and enzymes present in biologic fluids.

E. 5′ (E)-vinyl-phosphonate (VP) modification

5′-vinyl-phosphonate modifications (metabolically stable phosphate mimics) have been reported to enhance the metabolic stability and the potency of oligonucleotides.

### III. Nucleic Acid Embodiments

In certain embodiments the size of a nucleic acid may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 nucleic acid residues or greater, and any range derivable therein.

The nucleic acids of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more modified nucleic acids. In some embodiments, the nucleic acid of the disclosure may be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) identical in sequence with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 contiguous nucleic acids, or any range derivable therein, of SEQ ID NOS:1-43.

In some embodiments, the nucleic acid may comprise nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 (or any derivable range therein) of SEQ ID NOS:1-43.

In some embodiments, the nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 (or any derivable range therein) contiguous nucleic acids of SEQ ID NOS: 1-43.

In some embodiments, the nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 (or any derivable range therein) contiguous nucleic acids of SEQ ID NOS:1-43 that comprise at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity with one of SEQ ID NOS:1-43.

In some aspects there is a nucleic acid molecule starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 of any of SEQ ID NOS:1-43 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 (or any derivable range therein) contiguous nucleotides of any of SEQ ID NOS:1-43.

The nucleotide as well as the protein, polypeptide, and peptide sequences for various genes have been previously disclosed, and may be found in the recognized computerized databases. Two commonly used databases are the National Center for Biotechnology

Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” or “nucleic acid” are used interchangeable and refer to a nucleic acid molecule that may be recombinant or synthetically synthesized. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that may encode a protein, polypeptide, or peptide, or a region thereof, or a complement to a protein, peptide or region of a protein, such as a region of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 contiguous amino acids of a region or complement of a region of a gene or mRNA (either coding or non-coding region).

As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length, may be combined with other nucleic acid sequences, such as promoters, polyadenylation signals, additional restriction enzyme sites, multiple cloning sites, coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5x sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

B. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or part of one or more of the CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

IV. Methods of Treatment

The nucleic acids of the disclosure may be used to treat diseases by increasing translation of a cellular RNA. It is contemplated that the nucleic acids of the disclosure may be used to treat AIDS, autoimmune diseases (rheumatoid arthritis, multiple sclerosis, diabetes—insulin-dependent and non-independent, systemic lupus erythematosus and Graves disease); cancer (e.g., malignant, benign, metastatic, precancer); cardiovascular diseases (heart disease or coronary artery disease, stroke—ischemic and hemorrhagic, and rheumatic heart disease); diseases of the nervous system; and infection by pathogenic microorganisms (Athlete's Foot, Chickenpox, Common cold, Diarrheal diseases, Flu, Genital herpes, Malaria, Meningitis, Pneumonia, Sinusitis, Skin diseases, Strep throat, Tuberculosis, Urinary tract infections, Vaginal infections, Viral hepatitis); inflammation (allergy, asthma); prion diseases (e.g., CJD, kuru, GSS, FFI), Abdominal Adhesions; Anal Abscess; Brain Abscess; Peritonsillar Abscess; Absence Seizures; Achalasia; Acne; Acoustic Neuroma; Acquired Immunodeficiency Syndrome (AIDS); Acrochordon; Actinic Keratosis; Adenocarcinoma of the Lung; ADHD; Adult-Onset Diabetes; Aero-Otitis; Age Spots; Age-Related Hearing Loss; Age-Related Macular Degeneration; Age-Related Vision Change (Presbyopia); Agoraphobia; Alcohol Withdrawal; Alcoholism; Allergen Immunotherapy; Allergic Rhinitis; Allergies; Alopecia (Areata, Hereditary-Patterned, and Traumatic); Altitude Sickness; Alzheimer's Disease; Amaurotic Familial Infantile Idiocy; Amblyopia; Amenorrhea; Amyloidosis; Amyotrophic Lateral Sclerosis (ALS); Anaphylaxis; Androgenetic Alopecia; Anemia (Aplastic, Hemolytic, Pernicious, and Sickle Cell); Angina; Angiomas, Spider; Angioplasty; Ankylosing Spondylitis; Anorexia Nervosa; Anovulatory Bleeding; Antibiotic-Associated Diarrhea; Antiphospholipid Antibody Syndrome; Antisocial Personality Disorder; Anus Fissure, Fistula, Hemorrhoids, Anus Itch, Stricture; Anxiety Disorders (Generalized, Obsessive-Compulsive Disorder, Panic Disorder, Phobia, and Post-Traumatic Stress Disorder); Aortic Aneurysm; Aortic Arch Syndrome; Appendicitis; Arrhythmias, Cardiac; Arteritis, Takayasu's; Arthritic Diseases (Ankylosing Spondylitis, Gout, Infectious, Juvenile, Osteoarthritis, Pseudogout, Psoriatic Arthritis, and Rheumatoid); Asbestosis; Ascending Cholangitis; Asteatotic Eczema; Asthma; Astigmatism; Asymptomatic Bacteriuria; Ataxia, Friedreich's; Atherosclerosis; Athlete's Foot; Atopic Dermatitis; Atrial Fibrillation; Atrophic Vaginitis; Attention-Deficit Hyperactivity Disorder; Autism; Autoimmune Diseases (Celiac Disease, Crohn's Disease, Diabetes Mellitus, Type 1 (Insulin-Dependent; Juvenile-Onset), Diabetes Mellitus, Type 2 (Non-Insulin-Dependent; Adult-Onset), Graves' Disease, Hyperthyroidism, Immune Thrombocytopenic Purpura, Lupus, Myasthenia Gravis, Polyarteritis Nodosa, Rheumatoid Arthritis, Scleroderma, Takayasu's Arteritis, and Ulcerative Colitis); B12 Deficiency; Bacillary Dysentery; Bacterial Gastroenteritis; Bacterial Vaginosis; Balanitis; Baldness, Hereditary-Patterned; Barber's Itch; Barotitis; Barotrauma; Bartholin's Gland Cyst; Basal-Cell Carcinoma; Bed-Wetting; Bedsores; Behcet's Syndrome; Bell's Palsy; Bends; Benign Prostatic Hyperplasia; Bile-Duct Diseases; Biliary Colic; Biopsy; Bipolar Disorder; Bladder conditions (Infection; Interstitial Cystitis; Prolapse; Urethritis; Urinary Incontinence; Urinary Tract Infection); Blepharitis; Blepharoptosis; Blighted Ovum; Friction Blisters; Blood Pressure, High; Boils; Bone diseases and conditions (Osteoporosis;Paget's Disease); Bone Yaws; Borderline Personality Disorder; Bornholm Disease; Botulism; Bowel Obstruction; Bradycardia; Bronchitis; Bulimia Nervosa; Bunion; Bursitis; C. Difficile Colitis; Calcaneal Apophysitis; Calcium Pyrophosphate Deposition Disease; Campylobacteriosis; Cancer; Candidiasis; Carbon-Monoxide Poisoning; Carbuncles; Cardiac Arrhythmias (Atrial Fibrillation, Bradycardia); Cardiomyopathy; Carpal Tunnel Syndrome; Cataracts; Cellulitis; Central Serous Retinopathy; Cerebral Palsy; Cerebromacular Degeneration; Cerumen Impaction; Cervicitis, Nabothian Cysts, Cervical Polyps, Cervical Warts; Chalazion; Chickenpox; Chlamydia; Chloasma; Cholangitis; Cholecystitis; Cholesteatoma; Chondromalacia; Chorea; Choroidal Melanoma; Chronic Bronchitis; Chronic Fatigue Syndrome; Chronic Hepatitis; Chronic Leukemia; Chronic Obstructive Pulmonary Disease; Chronic Otitis Media; Cirrhosis; Cluster Headache; Cogan's Syndrome; Cold, Common; Colic, Biliary; Pseudomembranous Colitis, Ulcerative Colitis, Collapsed Lung; Collarbone Fracture; Coma; Complex Regional Pain Syndrome; Congestive Heart Failure; Conjunctivitis; Constipation; Contact Dermatitis; Conversion Disorder; COPD; Cornea Abrasion, Cornea Keratitis; Corns; Coronary Artery Disease; Creutzfeldt-Jakob Disease; Crossed Eyes; Croup; Cryptorchidism; Cystic Fibrosis; Interstitial Cystitis; Cystocele; Cysts; Cytomegalovirus infection; Dacryocystitis; Dandruff; Decompression Sickness; Decubitus Ulcers; Delirium Tremens; Delusional Disorder; Dementia; Depressive Disorders (Bipolar Disorder, Dysthymia, Major Depression, Manic Depression, Postpartum Depression); Dermatitis; Dermatofibroma; Dermatomyositis; Detached Retina; Developmental Dysplasia of the Hip; Deviated Septum; Devil's Grip; Diabetes (Gestational Diabetes; Type 1 Diabetes (Insulin-Dependent; Juvenile); Type 2 Diabetes (Non-Insulin-Dependent; Adult-Onset); Hypoglycemia, Ketoacidosis, Nephropathy, Neuropathies, Retinopathy) Antibiotic-Associated Diarrhea; Diplopia; Herniated Disk; Dislocated Lens; Hip Dislocation (Developmental); Diverticulitis; Diverticulosis; Dizziness; Doerderland's Vaginitis; Double Vision; Down Syndrome; Drooping Eyelid; Dry Skin; Sun-Damaged Skin; Dry-Eye Syndrome; Duck-Foot; Dysautonomia, Familial; Dysfunctional Uterine Bleeding; Dyslexia; Dyspareunia; Dysthymia; Dysuria; Eating Disorders (Anorexia Nervosa, Bulimia Nervosa); Eclampsia; Eczema; Edema; Emphysema; Encephalitis; Encopresis; End-Stage Renal Disease; Endocarditis; Endometriosis; Endophthalmitis; Endoscopy; Enlarged Prostate; Enuresis; Epidemic Benign Dry Pleurisy; Epididymitis; Epiglottitis; Epilepsy; Epistaxis; Erectile Dysfunction; Erythema Infectiosum; Esophagitis; Esophagus Achalasia; Esophagitis; Essential Hypertension; Essential Tremor; Ewing's Sarcoma; Familial Dysautonomia; Farsightedness; Febrile Seizures; Fecal Incontinence; Fever; Fever-Induced Seizures; Fibroids; Fibromyalgia; Fifth Disease; Filiform Warts; Flat Warts; Flatulence; Flu; Focal Seizures; Food Allergy; Food Poisoning; Forefoot Neuroma; Fragile X Syndrome; Friction Blisters; Friedreich's Ataxia; Frostbite; Fungal Infections (Athlete's Foot, Brain Abscess, Infectious Arthritis, Jock Itch, Onychomycosis, Ringworm, Swimmer's Ear, Tinea Cruris, Tinea Unguium, Tinea Versicolor); Furuncle; Gallstones; Gardnerella Vaginitis; Gastritis; Gastrocnemius Strain; Gastroenteritis; Gastroesophageal Reflux Disease; Gastrointestinal Amebiasis; Generalized Anxiety Disorder; Generalized Barotrauma; Genital Herpes; Genital Warts; GERD; Germ Cell Tumors, Extragonadal; Giant Cell Arteritis; Giardiasis; Glaucoma; Glomerulonephritis; Gluten-Sensitive Enteropathy; GM2 Gangliosidosis; Gonorrhea; Gout; Grand Mal Seizures; Graves' Disease; Graves' Ophthalmopathy; Guillain-Barré Syndrome; Hammertoe; Hay Fever; Headache; Hearing Loss; Heart Attack; Heat Stroke; Heel Spur; Heloma; Spider Hemangiomas; Hematoma; Hematuria; Hemochromatosis; Hemolytic Anemia; Hemophilia; Hemorrhagic Stroke; Subarachnoid Hemorrhagic Stroke; Hemorrhoids; Hepatitis A; Hepatitis B; Hepatitis C; Hereditary-Patterned Baldness; Hernia; Herniated Disk; High Blood Pressure; High Cholesterol; Hirsutism; Histiocytosis X; HIV/AIDS; Hordeolum; Human Papilloma Virus (HPV); Huntington's Disease; Hydatidiform Mole; Hydrocephalus; Hyperactivity; Hypercholesterolemia; Hyperkeratosis; Hyperopia; Hypertension; Ocular Hypertension; Secondary Hypertension; Hypertensive Retinopathy; Hyperthermia; Hyperthyroidism; Hypochondriasis; Hypoglycemia; Hypoparathyroidism; Hypothyroidism; IBS; ICD; Ichthyosis; Immune Thrombocytopenic Purpura; Impetigo; Impotence; Incontinence; Infantile Ganglioside Lipidosis; Infectious Arthritis; Infectious Mononucleosis; Infertility; Inflammatory Bowel Disease; Inguinal Hernia; Insomnia; Intercerebral Hemorrhage; Interdigital Neuroma; Intermetatarsal Neuroma; Intermittent Claudication; Interstitial Cystitis; Intestinal Obstruction; Iron Deficiency; Irritable Bowel Syndrome; Juvenile Arthritis; Kaposi's Sarcoma; Kawasaki Syndrome; Keloids; Keratitis; Actinic Keratosis; Labyrinthitis; Lactose Intolerance; Lacunar Stroke; Langerhans' Cell Histiocytosis; Laryngitis; Laryngotracheitis; Lateral Epicondylitis; Latex Allergy; Lazy Eye; Lead Poisoning; Intermittent Claudication; Restless Legs Syndrome; Shin Splints; Leg Strain; Cataract; Dislocated Lens; Leukemia; Lice; Lichen Simplex Chronicus; Cirrhosis; Hepatitis; Liver Spots; Lockjaw; Lou Gehrig's Disease; Lupus Erythematosus, Systemic; Lyme Disease; Lymphedema; Lymphoma; Macular Degeneration; Malabsorption Syndromes; Malaria; Male Pattern Baldness; Malignant Hyperthermia; Manic Depression; Marfan's Syndrome; Mastoiditis; Measles; Meckel's Diverticulum; Melasma; Meniere's Disease; Meningitis; Menopause; Mental Retardation; Phenylketonuria; Migraine; Miscarriage; Mitral-Valve Prolapse; Mittelschmerz; Molar Pregnancy; Molluscum Contagiosum; Mononucleosis; Morton's Neuroma; Mosaic Warts; Motor Tics; Mucocutaneous Lymph Node Syndrome; Multiple Sclerosis; Mumps; Muscular Dystrophy; Musculoskeletal Disorders (Fibromyalgia, Giant Cell Arteritis, Gout, Infectious Arthritis, Muscular Dystrophy, Myositis, Osteoarthritis, Osteoporosis, Paget's Disease Of Bone, Polymyalgia Rheumatica, Pseudogout, Reflex Sympathetic Dystrophy, Rheumatoid Arthritis, Scleroderma, Systemic Lupus Erythematosus, Tendonitis); Myasthenia Gravis; Myocardial Infarction; Myocarditis; Myopia; Myositis; Nail Felon; Onycholysis; Onychomycosis; Paronychia; Subungual Hematoma; Narcolepsy; Nasal Polyps; Nausea; Nearsightedness; Needle Biopsy; Nephrectomy; Nephroblastoma; Nephrolithiasis; Nephropathy, Diabetic; Neuritis, Retrobulbar; Neuroblastoma; Neuromuscular Disorders; Neuropathies; Guillain-Barre Syndrome; Retrobulbar; Nevi; Nevus Flammeus; Nevus Simplex; Nocturnal Enuresis; Non-Tropical Sprue; Obesity; Obsessive-Compulsive Disorder; Occupational Hearing Loss; Ocular Hypertension; Ocular Rosacea; Onycholysis; Onychomycosis; Glaucoma; Retrobulbar Neuritis; Optic Nerve Swelling; Orbit Fracture; Orchitis; Osgood-Schlatter Disease; Osteoarthritis; Osteoporosis; Osteosarcoma; Otitis Externa; Otitis Media; Chronic Otitis Media; Otosclerosis; Ototoxicity; Pelvic Inflammatory Disease; Polycystic Ovary Syndrome; Painful-Bladder Syndrome; Pancreatitis; Panic Disorder; Papilledema; Paraphimosis; Parkinson's Disease; Paronychia; Partial Seizures; PCL Injuries; Pedunculated Warts; Pelvic Relaxation; Paraphimosis; Peyronie's Disease; Peptic Ulcer; Perforated Eardrum; Pericarditis; Perimenopause; Peripheral Vascular Disease; Peritonsillar Abscess; Persistent Vegetative State; Personality Disorders; Petit Mal Seizures; Peyronie's Disease; Pharyngitis; Pharynx Cancer; Phenylketonuria; Phimosis; Phobia; Photosensitivity; Pigmentation Disorders (Chloasma, Melasma, Vitiligo); Piles; Pinkeye; Pityriasis Rosea; PKU; Plague; Plantar Fasciitis; Plantar Warts; Plantaris Strain; Pleurisy; Pleurodynia; PMS; Pneumoconiosis; Pneumonectomy; Pneumonia; Pneumothorax; Lead Poisoning; Polio; Poliomyelitis; Polyarteritis Nodosa; Polychondritis; Polymyalgia Rheumatica; Polymyositis; Colonic Polyps; Nasal Polyps; Vocal Cord Polyps; Port-Wine Stain; Post-Polio Syndrome; Postinfectious Thrombocytopenia; Postpartum Depression; Preeclampsia; Pregnancy-Induced Hypertension; Premenstrual Syndrome; Pressure Sores; Primary Sclerosing Cholangitis; Prolapse; Enlarged Prostate; Acute Prostatitis; Chronic Prostatitis; Pruritus Ani; Pseudogout; Psoriasis; Psoriatic Arthritis; Ptosis; Pulseless Disease; Pyelonephritis; Quadriceps Strain; Quinsy; Rash; Raynaud's Phenomenon; Rectal Itch; Rectocele; Reflex Sympathetic Dystrophy; Renal Failure; Respiratory Disorders Respiratory Syncytial Virus; Retina Detachment; Retinitis Pigmentosa; Retinopathy; Retrobulbar Neuritis; Reye's Syndrome; Rhabdomyosarcoma; Rheumatoid Arthritis; Allergic Rhinitis; Viral Rhinitis (Common Cold); Riley-Day Syndrome; Ringworm; Rocky Mountain Spotted Fever; Rosacea; Rubeola; Mumps; Salivary Gland Disorders; Salmon Patch; Sarcoidosis; Scabies; Scarlet Fever; Scars; Schizophrenia; Schizotypal Personality Disorder; Sciatica; Scleritis; Scleroderma; Scoliosis; Sebaceous Cysts; Seborrhea; Seborrheic Keratoses; Secondary Hypertension; Seizures; Sexual Dysfunction; Sexually Transmitted Diseases; Shigellosis; Shingles; Sialadenitis; Sialadenosis; Sialolithiasis; Sickle-Cell Anemia; Siderosis; Silicosis; Sinus Cancer; Sjögren's Syndrome; Sleep Disorders; Smallpox; Social Anxiety Disorder; Solar Lentigo; Somatoform Disorders (Hypochondriasis, Somatization Disorder); Somnambulism; Spastic Colon; Spider Veins; Spina Bifida; Spinal Cord Trauma; Spontaneous Abortion; Stasis Dermatitis; Strabismus; Strep Throat; Streptococcal Toxic Shock Syndrome; Stroke; Subarachnoid Hemorrhage; Transient Ischemic Attack; Stuttering; Subungual Hematoma; Sun Allergy; Sun-Damaged Skin; Sylvest's Disease; Systemic Lupus Erythematosus; Systemic Sclerosis; Tachycardia; Takayasu's Arteritis; Tay-Sachs Disease; Tear-Duct Infection; Telogen Effluvium; Temporal Arteritis; Tendonitis; Tennis Elbow; Tension Headache; Testicular Torsion; Undescended Testicles; Tetanus; Thrombocytopenia; Thrombophlebitis; Thrombotic Stroke; Tinea; Tinnitus; Tonsillitis; Torsional Deformities; Toxemia Of Pregnancy; Toxic Shock Syndrome, Streptococcal; Toxoplasmosis; Trichomoniasis; Trigeminal Neuralgia (Tic Douloureux); Tuberculosis; Tylosis; Ulcer; Urethritis; Urinary Tract disorders and conditions; Uroliniasis; Urticaria; Uterine disorders; Uterine Prolapse; Uveitis; Vaginitis; Bacterial (Gardnerella) Vaginosis; Varicella; Varices, Esophageal; Varicose Veins; Vascular Disorders (Hypertension, Intermittent Claudication, Peripheral Vascular Disease, Polyarteritis Nodosa, Raynaud's Phenomenon, Takayasu's Arteritis, Thrombophlebitis, Vasculitis, Wegener's Granulomatosis); Vein Inflammation; Varicose Veins; Vertigo; Vestibular Schwannoma; Viral Rhinitis; Vitamin B12 Deficiency; Vitiligo; Vocal Tics; Vocal-Cord Disorders; Common Warts; Genital Warts; Plantar Warts; Water On The Brain; Wax Blockage Of Ear Canal; Esophageal Webs; Werlhof s Disease; Wrinkles; Yersinia Pestis Infection. It is contemplated that such diseases can be diagnosed or treated using a nucleic acids of the invention that correspond to miRNAs.

It is also contemplated that the nucleic acids of the disclosure may be useful in treating cancers. In some embodiments, the nucleic acid of the disclosure treats a cancer by increasing translation of a tumor suppressor. Cancers that may be treated in the methods of the disclosure include cancers of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Moreover, the nucleic acid may be used to treat precancers, such as metaplasia, dysplasia, and hyperplasia.

### V. Administration of Therapeutic Compositions

The therapy provided herein may comprise administration of a combination of therapeutic agents. The therapies may be administered in any suitable manner known in the art. Embodiments of the disclosure relate to compositions and methods comprising therapeutic compositions. Different therapies or therapeutic molecules, such as one or more nucleic acids described herein may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VI. Pharmaceutical Preparations

In one aspect, the methods disclosed herein can include the administration of pharmaceutical compositions and formulations comprising nucleic acid agents capable of modulating the activity or expression level of at least one gene or mRNA, such as an endogenously expressed gene or mRNA, in a cell.

In certain embodiments, the compositions are formulated with a pharmaceutically acceptable carrier. The pharmaceutical compositions and formulations can be administered parenterally, topically, by direct administration into the gastrointestinal tract (e.g., orally or rectally), or by local administration, such as by aerosol or trans-dermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., oligonucleotides) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

In certain embodiments, the pharmaceutical compositions and formulations are administered by in intranasal, intraocular and intravaginal routes including suppositories, insufflation, powders and aerosol formulations (for examples of steroid inhalants, see e.g., Rohatagi (1995) J. Clin. Pharmacol. 35:1 187-1193; Tjwa (1995) Ann. Allergy Asthma Immunol. 75: 107-1 11). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In certain embodiments, the pharmaceutical compositions and formulations are delivered trans-dermally, by a topical route, formulated as applicator sticks, solutions, suspensions, emulsions, gels, creams, ointments, pastes, jellies, paints, powders, and aerosols.

In certain embodiments, the pharmaceutical compositions and formulations are delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In certain embodiments, the pharmaceutical compounds and formulations are lyophilized. Stable lyophilized formulations comprising an inhibitory nucleic acid can be made by lyophilizing a solution comprising a pharmaceutical and a bulking agent, e.g., mannitol, trehalose, raffinose, and sucrose or mixtures thereof. A process for preparing a stable lyophilized formulation can include lyophilizing a solution about 2.5 mg/mL nucleic acid, about 15 mg/mL sucrose, about 19 mg/mL NaCl, and a sodium citrate buffer having a pH greater than 5.5 but less than 6.5. See, e.g., U.S. 20040028670.

In certain embodiments, the pharmaceutical compositions and formulations are delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46: 1576-1587.

### VII. Kits

Certain aspects of the present disclosure also concern kits containing compositions of the disclosure or compositions to implement methods of the invention. In some embodiments, kits can be used to evaluate expression levels of protein or mRNA. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or translation activators of the disclosure for prognostic or diagnostic applications are included as part of the disclosure. Specifically contemplated herein are any such molecules corresponding to any nucleic acid identified herein, which includes nucleic acid primers/primer sets and probes that are identical to or complementary to all or part of a nucleic acid disclosed herein, which may include noncoding as well as coding portions of mRNA and genes.

In certain aspects, negative and/or positive control nucleic acids, probes, and inhibitors are included in some kit embodiments.

It is contemplated that any method or composition described herein can be implemented with respect to any other method or composition described herein and that different embodiments may be combined. The claims originally filed are contemplated to cover claims that are multiply dependent on any filed claim or combination of filed claims.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Antisense Translation Activating RNAs

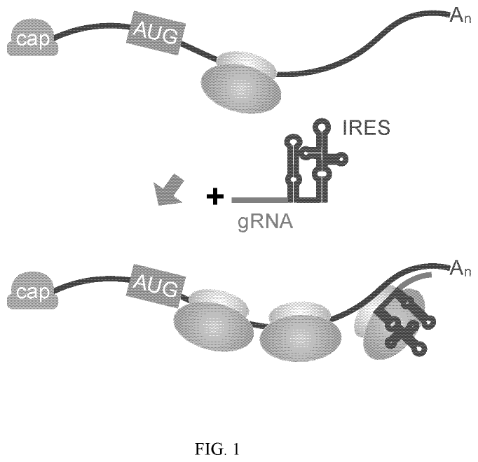

The inventors designed an anti-sense translation activating RNA that comprises an anti-sense guide RNA (targeting region) that recognizes and binds target mRNA and an IRES RNA (ribosome binding site) to recruit ribosomes for increasing translation efficiency of targeted mRNA (FIG. 1). For the guide RNA design, the inventors first adopted designs from the CIRTS system (PMID: 31230714) to make the 40-nt guide RNA (gRNA). To choose the IRES RNA, the inventors first decided on the EMCV (encephalomyocarditis virus) and HCV (hepatitis C virus) IRES, which are broadly used and studied. The inventors also chose the PTV-1 (porcine teschovirus type 1) IRES, which is very similar to HCV IRES both functionally and structurally (PMID: 15078929). The inventors are also interested in another kind of virus IRES, CrPV (cricket paralysis virus) IRES, which directly recruits ribosomes with no requirement of any initiation factor or even initiator tRNA (PMID: 12470947).

Next, the inventors referred to the crystal structure of IRES bound to 80s ribosome (PMID: 18468443) to find out the 3′ end of IRES RNA is usually buried into ribosome, which helps it to initiate downstream RNA translation. Thus, the inventors chose to fuse the guide RNA to the more exposed 5′ end of IRES.

To make it convenient to test the anti-sense translation activating RNA, the inventors used the dual-luciferase reporter, which encodes two luciferases—firefly luciferase and Renilla luciferase, with two distinct promotors. The inventors sought to increase firefly luciferase expression and leave Renilla as a control to normalize transfection and cell status.

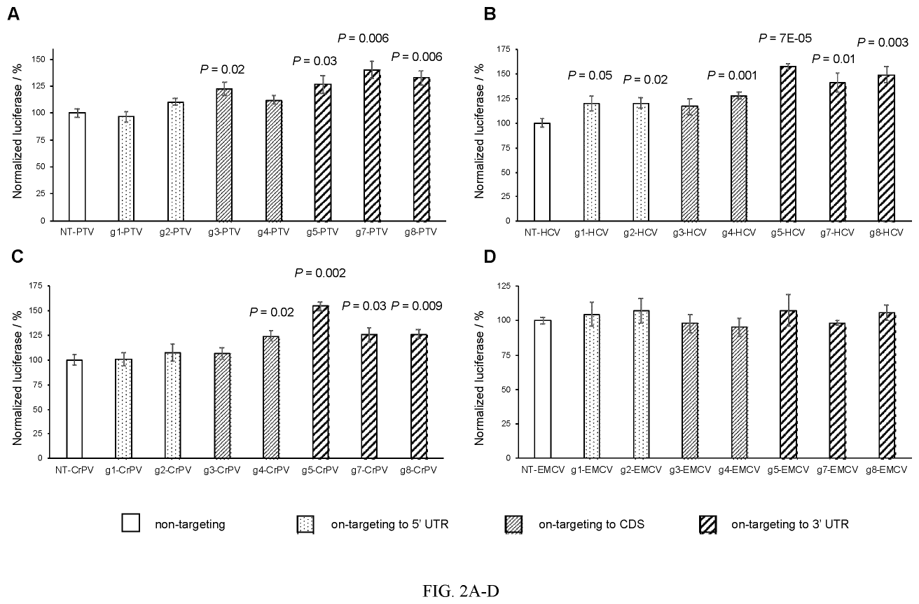

After the conceptual design, the inventors constructed gRNA-IRES with EMCV, HCV, PTV-1 or CrPV IRES. The guide RNAs (g1 to g7 and g8) are reversely complementary to either 5′ UTR, CDS, or 3′ UTR of the firefly luciferase mRNA to specifically increase local concentration of ribosomes near this mRNA. The inventors compared the luminescence signal ratio, firefly to Renilla luciferase, of on-target gRNA-IRES with that of non-target IRES control in HEK293T cells (FIG. 2A-D). The screening results of PTV-1, HCV and CrPV indicate these three kinds of gRNA-IRES could increase translation of targeted mRNA, especially when gRNAs are antisense of the 3′ UTR of mRNA. Besides, the gRNA-EMCV, which couldn't increase firefly luciferase level, serve as a negative control that proves the correct IRES RNA part is important for this system to function at a high level of translational activation (FIG. 2D).

The relative low increase for luciferase reporter mRNA could be expected since the reporter mRNA was engineered to be highly translated, which means the endogenous ribosome-binding could be near saturation, and this system functions through recruiting more endogenous ribosomes without any extra translational machinery added. These luciferase reporter experiments encouraged the inventors to test the system with endogenous targets.

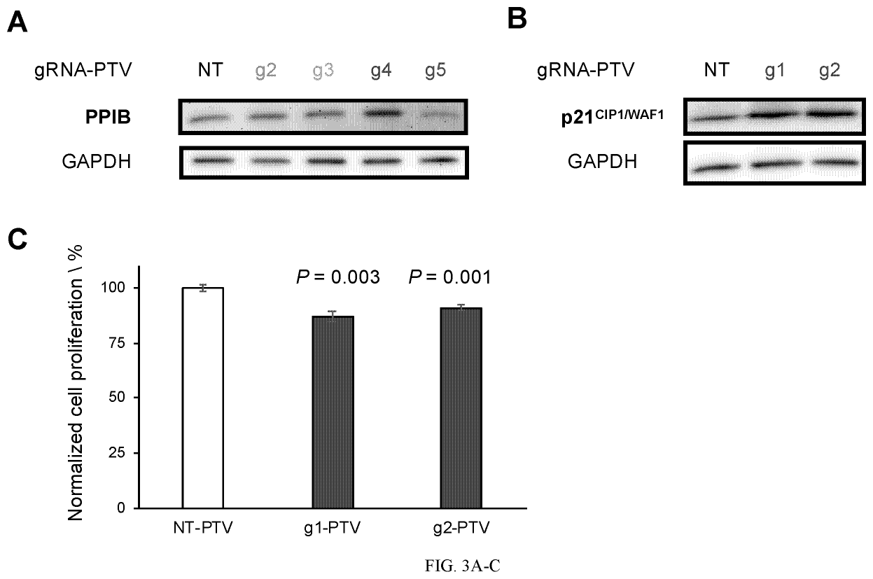

The inventors selected PPIB (Peptidylprolyl Isomerase B) as the first endogenous target and used the gRNA-PTV-1 IRES tool to attempt to boost protein levels in HEK293T cells. By western blotting, the inventors could detect significant higher protein amount when adding 3′ UTR targeting gRNA (g4) (FIG. 3A) compared to non-targeting control (NT). At the same time, the fact that gRNA targeting 5′ UTR (g2) and CDS regions (g3) of PPIB mRNA didn't work as efficient as gRNA for 3′ UTR, is consistent with the luciferase assay results to provide clues for gRNA design.

Before going further, the inventors decided to check if the protein level increase caused by gRNA-IRES could affect the cell function. The inventors chose a cell cycler inhibitor, p21^{CIP1/WAF1} which functions as anti-proliferative effector in normal cells, to exam if the cell proliferation rate would be decreased by p21^{CIP1/WAF1} targeted gRNA-IRES treatment. The western blotting proved the gRNA-PTV-1 IRES could increase p21^{CIP1/WAF1} in HEK293T cells at the first place (FIG. 3B), and then the inventors tested cell proliferation by MTS assay (FIG. 3C). This mild growth attenuation of cell proliferation is comparable to p21^{CIP1/WAF1} plasmid and other methods induced (PMID: 27215384, PMID: 25307521, PMID 17085592). With this result, the inventors decided to test whether this method could boost protein levels from other disease-related targets.

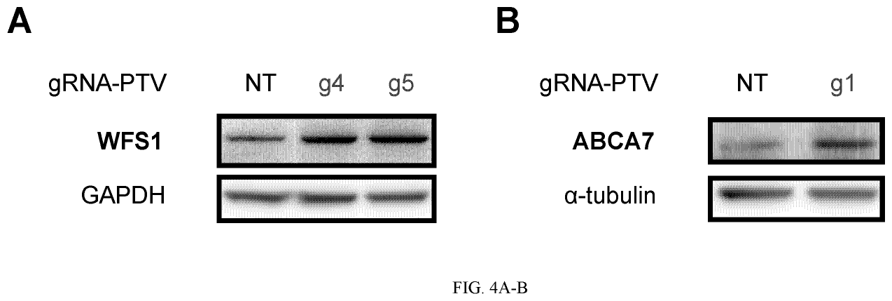

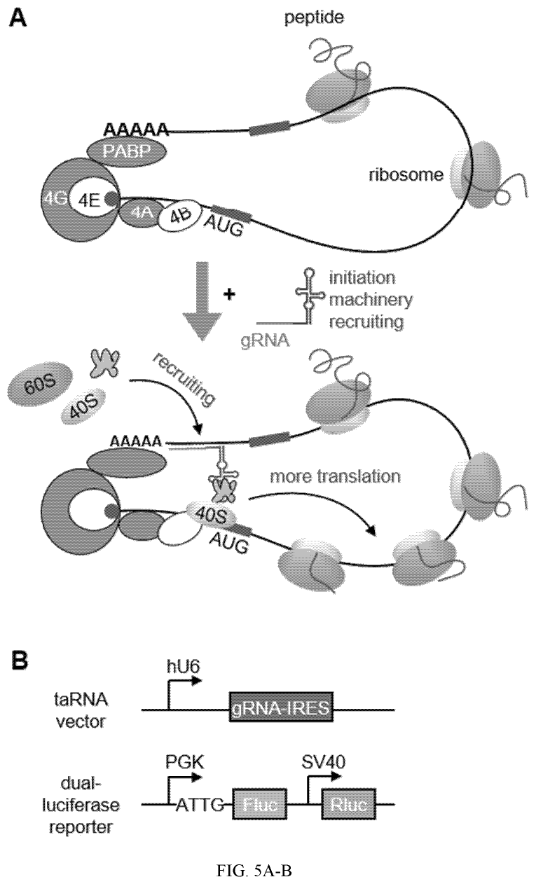

Wolfram syndrome is a rare autosomal recessive disorder characterized by juvenile-onset diabetes mellitus, diabetes insipidus, optic nerve atrophy, hearing loss, and neurodegeneration (PMID: 26742931). The mutated WFS1 locus is the main cause for this disorder but patients still have one functional allele. It was hypothesized that the translation activators could help to relieve patients' symptom by increasing the intact WFS1 protein level to restore its function. As the fundamental evidence, the inventors designed gRNAs for WFS1 mRNA with PTV-1 IRES and tested efficacy for protein level increase in HEK293T cells. In the western blots, the inventors could also detect higher protein amount by treatment (g4 and g5) compared to control (NT) (FIG. 4A). The inventors would further test the system in the patient cell model (PMID: 24556864).

The lost-of-function mutations in one allele of ABCA7 has been associated with the higher risk for late-onset Alzheimer's Disease (PMID 21460840). Recently, the researchers have provided insights into the mechanisms that ABCA7 haplodeficiency may cause AD by impairing the microglial immune responses (PMID: 31690660). Providing therapies to raise functional ABCA7 protein level in ABCA7 haplodeficient population could possibly help lower their risk for Alzheimer's Disease. Thus, the inventors designed guide RNA-IRES using PTV-1 IRES for ABCA7, and tested the effect in HEK293T cells by western blotting, which showed higher ABCA7 protein amount with treatment (FIG. 4B), promising for further tests.

The data provided in this example provides a proof of concept and shows the efficiency of the antisense translation activating RNA by luciferase reporter and several endogenous targets. Therefore, this data demonstrates that the translational activators disclosed in this application can efficiently increase translation of disease relevant targets.

TABLE 1
SEQUENCES

| | | SEQ |
|---|---|---|
| | | ID |
| Description | Sequence | NO: |
| PTV-1 IRES | UACUUGGUUAUGAAUUCAUUGUAUUAACCCCUCUGA | 1 |
| sequence | AAGACCUGCUCUGGCGCGAGCUAAAGCGCAAUUGUC | |
| | ACCAGGUAUUGCACCAAUGGUGGCGACAGGGUACAG | |
| | AAGAGCAAGUACUCCUGACUGGGUAAUGGGACUGCA | |
| | UUGCAUAUCCCUAGGCACCUAUUGAGAUUUCUCUGG | |
| | GGCCCACCAGCGUGGAGUUCCUGUAUGGGAAUGCAG | |
| | GACUGGACUUGUGCUGCCUGACAGGGUCGCGGCUGG | |
| | CCGUCUGUACUUUGUAUAGUCAGUUGAAACUCACC | |
| HCV IRES | TCCCCTGTGAGGAACTACTGTCTTCACGCAGAAAGCGT | 2 |
| sequence | CTAGCCATGGCGTTAGTATGAGTGTCGTGCAGCCTCCA | |
| | GGCCCCCCCCTCCCGGGAGAGCCATAGTGGTCTGCGGA | |
| | ACCGGTGAGTACACCGGAATTGCCAGGACGACCGGGT | |
| | CCTTTCTTGGATCAATCCCGCTCAATGCCTGGAGATTT | |
| | GGGCGTGCCCCCGCGAGACTGCTAGCCGAGTAGTGTTG | |
| | GGTCGCGAAAGGCCTTGTGGTACTGCCTGATAGGGTGC | |
| | TTGCGAGTGCCCCGGGAGGTCTCGTAGACCGTGCACC | |
| EMCV IRES | GAGGGCCCGGAAACCTGGCCCTGTCTTCTTGACGAGCA | 3 |
| sequence | TTCCTAGGGGTCTTTCCCCTCTCGCCAAAGGAATGCAA | |
| | GGTCTGTTGAATGTCGTGAAGGAAGCAGTTCCTCTGGA | |
| | AGCTTCTTGAAGACAAACAACGTCTGTAGCGACCCTTT | |
| | GCAGGCAGCGGAACCCCCCACCTGGCGACAGGTGCCT | |
| | CTGCGGCCAAAAGCCACGTGTATAAGATACACCTGCA | |
| | AAGGCGGCACAACCCCAGTGCCACGTTGTGAGTTGGA | |
| | TAGTTGTGGAAAGAGTCAAATGGCTCTCCTCAAGCGTA | |
| | TTCAACAAGGGGCTGAAGGATGCCCAGAAGGTACCCC | |
| | ATTGTATGGGATCTGATCTGGGGCCTCGGTGCACATGC | |
| | TTTACATGTGTTTAGTCGAGGTTAAAAAAACGTCTAGG | |
| | CCCCCCGAACCACGGGGACGTGGTTTTCCTTTGAAAAA | |
| | CACGATGATAA | |
| CrPV IRES | AGCAAAAATGTGATCTTGCTTGTAAATACAATTTTGAG | 4 |
| sequence | AGGTTAATAAATTACAAGTAGTGCTATTTTTGTATTTA | |
| | GGTTAGCTATTTAGCTTTACGTTCCAGGATGCCTAGTG | |
| | GCAGCCCCACAATATCCAGGAAGCCCTCTCTGCGGTTT | |
| | TTCAGATTAGGTAGTCGAAAAACCTAAGAAATTTACCT | |
| | GCTACATTTCAAGA | |
| NT (lower case | tgacagcccacatggcattccacttatcactggcatccttTTATTUACUUGGUU | 5 |
| underline)-linker | AUGAAUUCAUUGUAUUAACCCCUCUGAAAGACCUGC | |
| (uppercase italic)- | UCUGGCGCGAGCUAAAGCGCAAUUGUCACCAGGUAU | |
| PTV-1 IRES | UGCACCAAUGGUGGCGACAGGGUACAGAAGAGCAAG | |
| (uppercase) | UACUCCUGACUGGGUAAUGGGACUGCAUUGCAUAUC | |
| | CCUAGGCACCUAUUGAGAUUUCUCUGGGGCCCACCAG | |
| | CGUGGAGUUCCUGUAUGGGAAUGCAGGACUGGACUU | |
| | GUGCUGCCUGACAGGGUCGCGGCUGGCCGUCUGUAC | |
| | UUUGUAUAGUCAGUUGAAACUCACC | |
| lux gRNA-1 | ggtggctttaccaacagtaccggattgccaagcttgggct | 6 |
| lux gRNA-2 | cgctgggcccttcttaatgtttttggcatcttccatggtg | 7 |
| lux gRNA-3 | atggcgctgggcccttcttaatgtttttggcatcttccat | 8 |
| lux gRNA-4 | ggtagaatggcgctgggcccttcttaatgtttttggcatc | 9 |
| lux gRNA-5 | caggtcgactctagactcgaggctagcgagctcgtttaaa | 10 |
| lux gRNA-7 | gctcagcggtggcagcagccaactcagcttcctttcgggc | 11 |
| lux gRNA-8 | tcatgtctgctcgaagcggccgccccaaggggttatgcta | 12 |
| PPIB-gRNA-2 | ccacaggcggaggcgaaagcagcccggacagctgaggccg | 13 |
| PPIB-gRNA-3 | caaggagcaccttcatgttgcgttcggagaggcgcagcat | 14 |
| PPIB-gRNA-4 | cctgcacagacggtcactcaaagaaagatgtccctgtgcc | 15 |
| PPIB-gRNA-5 | gaatgtgaggggagtgggtccgctccaccagatgccagca | 16 |
| p21-gRNA-1 | agagcgggcctttgaggccctcgcgcttccaggactgcag | 17 |
| p21-gRNA-2 | ggggggcagggggcggccagggtatgtacatgaggaggtg | 18 |
| WFS1-gRNA-4 | atggcaacatgcactggaagctcctcgtggcggaccatcc | 19 |
| WFS1-gRNA-5 | ttgtcggggtccacgcaatctacacatggtcgcaaggtct | 20 |
| ABCA7-gRNA-1 | attcccagggcctccccgcggccccgcaggggagggaggc | 21 |
| Non-targeting | tgacagcccacatggcattccacttatcactggcatccttTTATT | 22 |
| gRNA and linker | | |

### Example 2: Antisense Translation Activating RNAs Design Modifications

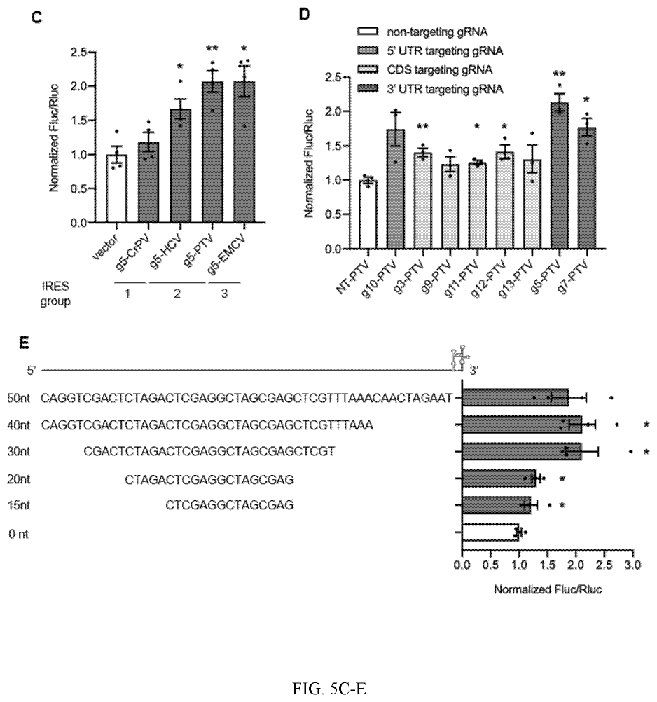

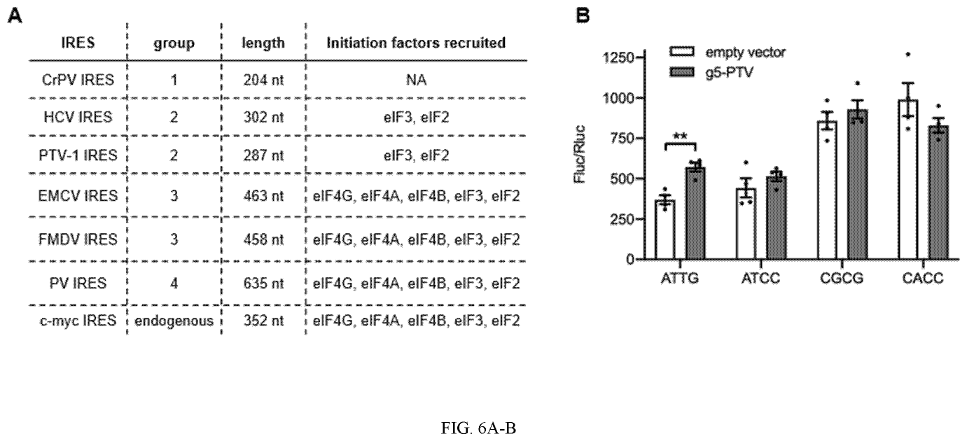

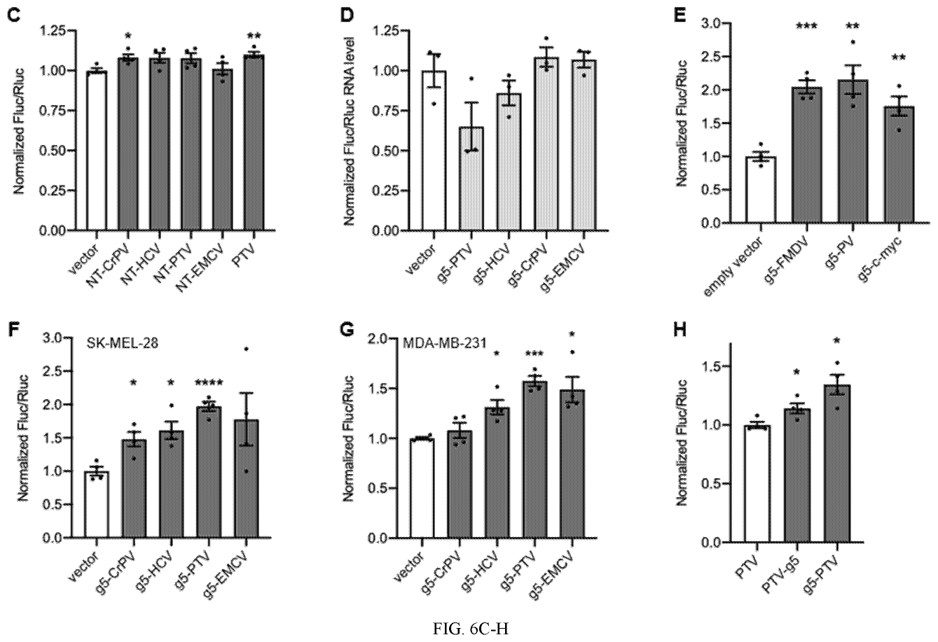

The inventors tested wither the dual-luciferase reporter, which now has a weaker Kozak sequence (position −4 to −1 is ATTG) than original one (CACC), could be engineered to be more responsive to translation initiation rate changes (FIG. 5B, 6B). With this engineered reporter, the inventors kept the g5 as the complementary gRNA for Firefly luciferase, and screened different groups of viral IRES and endogenous IRES as the recruiting domain of taRNA in HEK293T cells (FIG. 5C, 6E). The IRES from virus including HCV, PTV-1, EMCV, FMDV, PV and endogenous c-myc could all be effective to activate gene expression in taRNA construct, without significant interfere on target RNA amount (FIG. 6D). The inventors confirmed this screening in different cell lines and got similar results (FIG. 6F, 6G). Considering both the effectiveness and small size (FIG. 6A), the inventors chose PTV-1 IRES as the model to continue the validation.

With PTV-1 IRES as the recruiting domain, the inventors next explored the gRNA design. The inventors first investigated if the gRNA-landing position on mRNA transcripts would affect the performance of taRNA (FIG. 5D). Compared to the non-targeting control, the 3′-UTR targeting gRNAs (g5 and g7) gave an overall better effect than 5′-UTR targeting and CDS-targeting gRNAs, thus the inventors continued to use g5 as the gRNA domain for Firefly activating taRNA. Then, the inventors engineered gRNAs of different lengths that were reversely complementary to the mRNA sequence of g5-landing position (FIG. 5E). The inventors found that effective gRNAs were more than 20 nt and less than 50 nt in this test, and thus the inventors chose 40-nt as the length for gRNA design. Finally, the inventors checked if the orientation of gRNA domain and recruiting domain would be important for taRNA and determined that a taRNA with the gRNA on the 5′ performed better in this construction (FIG. 6H). To be more confident about the different controls being used, the inventors compared the controls in the same dual-luciferase assay and proved they all have similar effect or tiny changes compared to empty vector control (FIG. 6C). Adding the engineering designs from above, we decided on the 40-nt 3′-UTR targeting gRNA design and PTV-1 IRES as the translational machinery recruiting domain.

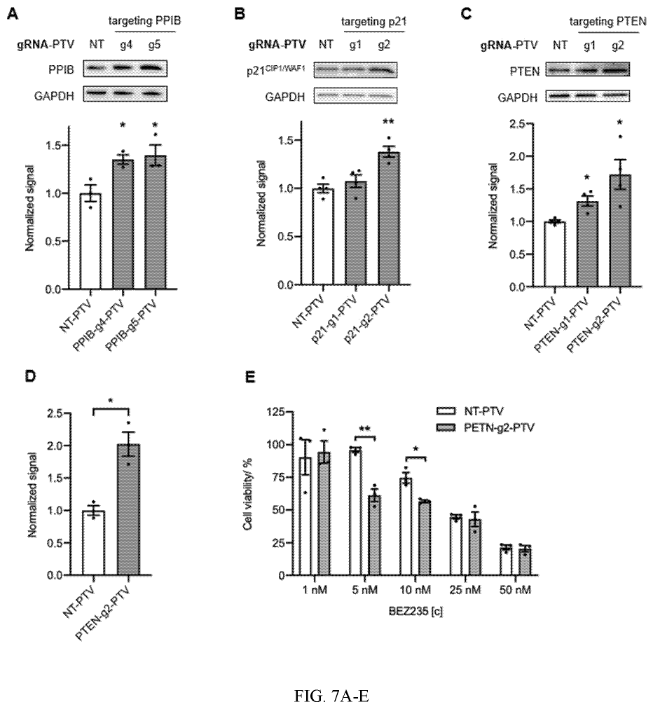

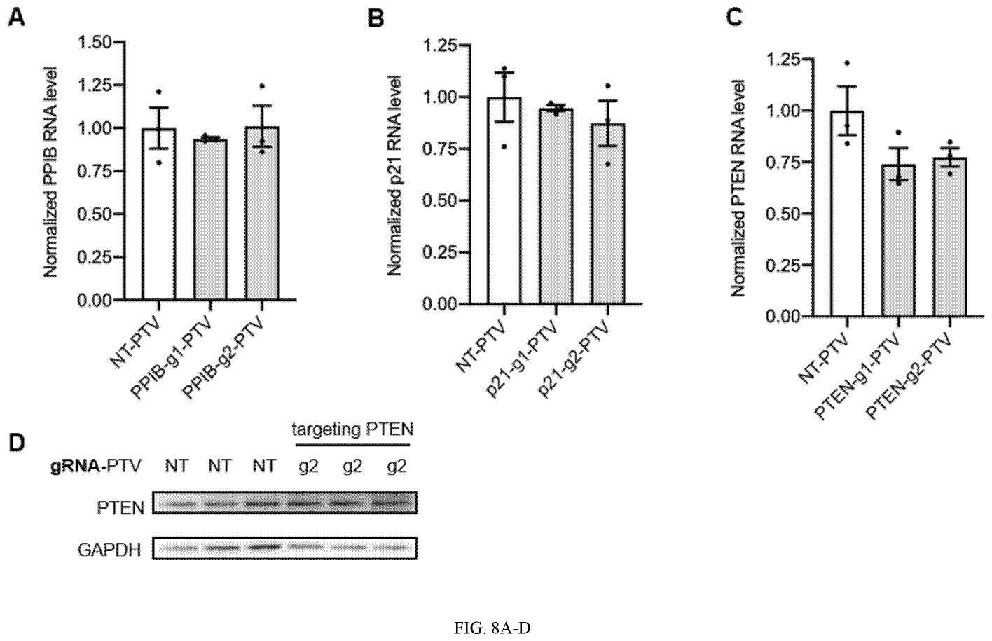

Having tested taRNA on reporter gene, the inventors continued to validate this tool on endogenous mRNAs in human cell lines. The inventors engineered at least two guide RNAs targeting 3′ UTR of endogenous transcripts, including NIB, p21^{CIP1/WAF1} and PTEN, and fused them with PTV-IRES to make individual taRNA. HEK293T cells transfected with respective taRNA were harvested and analyzed by immunoblotting to check the target gene expression at protein level. The inventors demonstrated that a taRNA construct was able to increase endogenous PPIB, p21^{CIP1/WAF1} and PTEN at protein level to about 1.5-fold (FIGS. 7A, 7B, 7C) but without interfering the RNA amount (FIGS. 8A, 8B, 8C).

To further prove the effectiveness of taRNAs, the inventors tested if PTEN-activating taRNA could sensitize triple-negative breast cancer cell line, MDA-MB-231 to pan PI3K/mTOR inhibitor, BEZ235. First proved by immunoblotting, the PTEN-targeting taRNA increased PTEN protein level (FIGS. 7D, 8D). Then the inventors treated MDA-MB-231 cells with either PTEN activating taRNA (PTEN-g2-PTV) or non-targeting taRNA control (NT-PTV), and added gradient dose of BEZ235 (FIG. 7E). Cancer cells treated with PTEN-activating taRNA indeed are more sensitive to lower dose of BEZ235 (5 nM and 10 nM) than with the non-targeting control. This finding is especially noteworthy, as dose-limiting toxicity of BEZ235 and other PI3K inhibitors have prevented their advancement through the clinical trial pipeline.

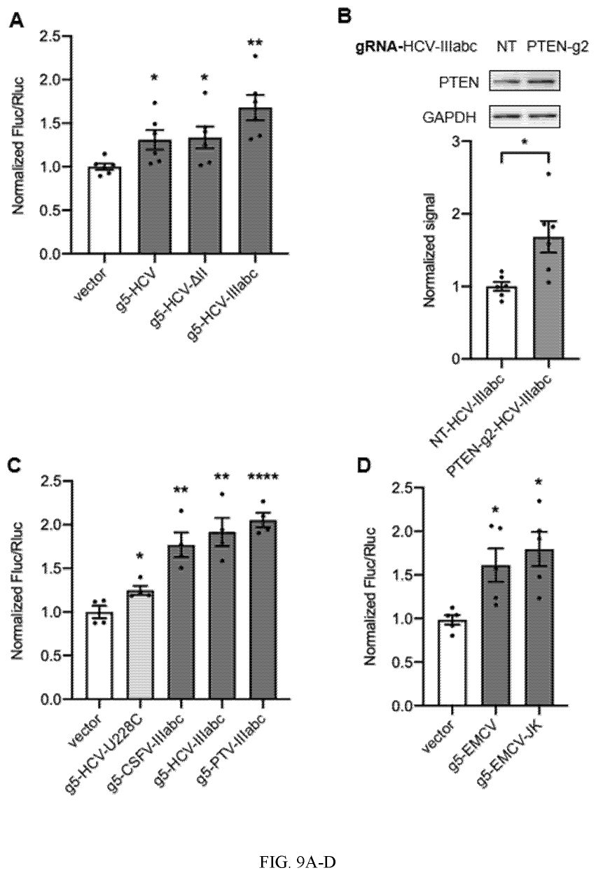

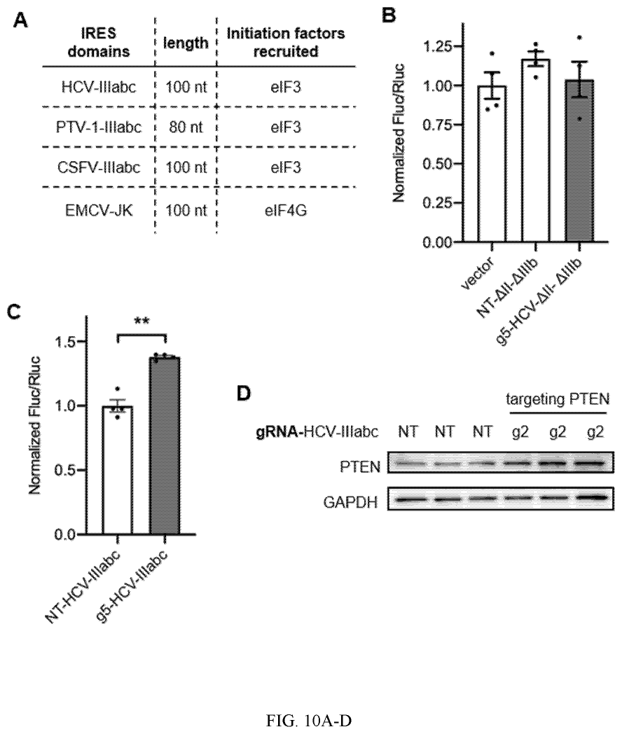

HCV IRES is a well-studied model for understanding viral IRES domains and their function in recruiting initiation machinery. Thus, the inventors truncated HCV IRES into different regions, where HCV-ΔII (removing domain II) still binds to both eIF3 and small ribosome 40S, HCV-IIIabc is known to bind eIF3 and HCV-ΔII-ΔIIIb (removing domain II and domain Mb) is known to recruit 40S [PMID: 11233977]. The inventors fused Firefly targeting gRNA, g5, to different truncated HCV IRES and tested their function on increasing Firefly expression FIGS. 9A, 10B). We found HCV-ΔII is as effective as full-length HCV and HCV-IIIabc had even better performance than full-length version, although ribosome-recruiting HCV-ΔII-ΔIIIb failed to increase Firefly signal. The effect of HCV-IIIabc taRNA was further proved by comparing to non-targeting control (FIG. 10C) and by increasing endogenous PTEN expression in HEK293T cells(FIG. 9B).

After uncovering the key aspects of the HCV IRES, the inventors examined if other eIF3-recruiting domains could also serve as taRNA function domain. The inventors chose IIIabc domains from CSFV IRES and PTV-1 IRES, which are in the same Group-2 as HCV. The inventors fused IIIabc domain from either Group 2 IRES with g5 guide RNA and both CSFV-IIIabc and PTV-IIIabc could increase Firefly expression as well as HCV-IIIabc (FIG. 9C). Furthermore, the inventors constructed the U228C-mutant of HCV-IIIabc in taRNA and found the single mutation abolishing eIF3 binding would significantly affect taRNA function (FIG. 9C), offering more evidence for that eIF3 recruiting itself could be effective as taRNA function domain.

Inspired by the eIF3 recruiting IIIabc domains of Group 2 IRES, the inventors turned to Group 3 IRES for their eIF4G recruiting domain, like the J-K region of EMCV IRES [doi:10.1038/nsmb.3280]. With similar dual-luciferase assay platform, the inventors confirmed the EMCV-JK region is also effective as the taRNA recruiting domain (FIG. 9D). These data confirm that both eIF3- and eIF4G-recuriting taRNAs can function, and that multiple groups and families of IRESs can be used as engineering starting points or taRNA development.

TABLE
IRES SEQUENCES

| | | SEQ |
|---|---|---|
| | | ID |
| Description | Sequence | NO: |
| PV-IRES | GACGCACAAAACCAAGTTCAATAGAAGGGGG | 41 |
| | TACAAACCAGTACCACCACGAACAAGCACTT | |
| | CTGTTTCCCCGGTGATGTCGTATAGACTGCTT | |
| | GCGTGGTTGAAAGCGACGGATCCGTTATCCGC | |
| | TTATGTACTTCGAGAAGCCCAGTACCACCTCG | |
| | GAATCTTCGATGCGTTGGTTAGCACTCAACCC | |
| | CAGAGTGTAGCTTAGGCTGATGAGTCTGGAC | |
| | ATCCCTCACCGGTGACGGTGTTCCAGGCTGCG | |
| | TTGGCGGCCTACCTATGGCTAACGCATGGGAC | |
| | GCTAGTTGTGAACAAGGTGTGAAGAGCCTATT | |
| | GAGCTACATAAGAATCCTCCGGCCCCTGAATG | |
| | CGGCTAATCCCAACCTCGGAGCAGGTGGTTCA | |
| | CAAACCAGTGATTGGCCTGTCGTAACGCGCA | |
| | AGTCCGTGGCGGAACCGACTACTTTGGGTGTC | |
| | CGTGTTTCCTTTTATTTTATTGTGGCTGCTTAT | |
| | GGTGACAATCACAGATTGTTATCATAAAGCG | |
| | AATTGGATTGGCCATCCGGTGAAAGTGAGAC | |
| | TCATTATCTATCTGTTTGCTGGATCCGCTCCAT | |
| | TGAGTGTGTTTACTCTAAGTACAATTTCAACA | |
| | GTTATTTCAATCAGACAATTGTATCATAATG | |
| FMDV- | CACGATTTAAGCAGGTTTCCACAACTGATAAA | 42 |
| IRES | ACTCGTGCAACTTGAAACTCCGCCTGGTCTTT | |
| | CCAGGTCTAGAGGGGTTACACTTTGTACTGTG | |
| | CTCGACTCCACGCCCGGTCCACTGGCGGGTGT | |
| | TAGTAGCAGCACTGTTGTTTCGTAGCGGAGCA | |
| | TGGTGGCCGTGGGAACTCCTCCTTGGTGACAA | |
| | GGGCCCACGGGGCCGAAAGCCACGTCCAGAC | |
| | GGACCCACCATGTGTGCAACCCCAGCACGGC | |
| | AACTTTTACTGCGAACACCACCTTAAGGTGAC | |
| | ACTGGTACTGGTACTCGGTCACTGGTGACAGG | |
| | CTAAGGATGCCCTTCAGGTACCCCGAGGTAAC | |
| | ACGGGACACTCGGGATCTGAGAAGGGGATTG | |
| | GGACTTCTTTAAAAGTGCCCAGTTTAAAAAGC | |
| | TTCTACGCCTGAATAGGCGACCGGAGGCCGG | |
| | CGCCTTTCCATTAC | |
| FMDV- | CAACCCTTGCCGCATCCACGAAACTTTGCCCA | 43 |
| IRES | TAGCAGCGGGCGGGCACTTTGCACTGGAACTT | |
| | ACAACACCCGAGCAAGGACGCGACTCTCCCG | |
| | ACGCGGGGAGGCTATTCTGCCCATTTGGGGAC | |
| | ACTTCCCCGCCGCTGCCAGGACCCGCTTCTCT | |
| | GAAAGGCTCTCCTTGCAGCTGCTTAGACGCTG | |
| | GATTTTTTTCGGGTAGTGGAAAACCAGCAGCC | |
| | TCCCGCGACGATGCCCCTCAACGTTAGCTTCA | |
| | CCAACAGGAACTATGACCTCGACTACGACTCG | |
| | GTGCAGCCGTATTTCTACTGCGACGAGGAGGA | |
| | GAACTTCTACCAGCAGCAGCAGCAGAGCGAG | |
| | CT | |

* * *

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein, to the extent that they provide exemplary procedural or other details supplementary to those set forth herein, are specifically incorporated herein by reference.

1. A chimeric nucleic acid comprising a targeting region and a translational activating region, wherein the translational activating region comprises at least one ribosome and/or translation factor binding site and wherein the targeting region comprises a region that is complementary to a target mRNA.

2. The nucleic acid of claim 1, wherein the targeting region comprises an antisense nucleic acid.

3. The nucleic acid of claim 1 or 2, wherein the mRNA comprises a mammalian mRNA.

4. The nucleic acid of claim 1 or 2, wherein the mRNA comprises a bacterial mRNA.

5. The nucleic acid of any one of claims 1-4, wherein the mRNA comprises an endogenously produced mRNA from a cell.

6. The nucleic acid of claim 5, wherein the cell comprises a prokaryotic or eukaryotic cell.

7. The nucleic acid of any one of claims 1-6, wherein the translational activating region comprises a ribosome binding site and wherein the ribosome binding site comprises a cap-independent ribosome binding site.

8. The nucleic acid of any one of claims 1-7, wherein the translational activating region comprises an internal ribosomal entry site (IRES) or a ribosome and/or translation factor binding fragment thereof.

9. The nucleic acid of claim 8, wherein the IRES comprises a Group 2 IRES or a ribosome and/or translation factor binding fragment thereof.

10. The nucleic acid of claim 9, wherein the IRES or IRES fragment comprises the IIIabc domain.

11. The nucleic acid of claim 8, wherein the IRES comprises a Group 4 IRES or a ribosome and/or translation factor binding fragment thereof.

12. The nucleic acid of claim 9, wherein the IRES or IRES fragment comprises the J-K region.

13. The nucleic acid of any one of claims 1-12, wherein the ribosome and/or translation factor binding site is from or is derived from a viral, mammalian, or plant ribosomal binding site.

14. The nucleic acid of any one of claims 1-13, wherein the translational activating region comprises an IRES from PTV-1, HCV, EMCV, or CrPV.

15. The nucleic acid of any one of claims 1-14, wherein the nucleic acid comprises 2 ribosome binding sites.

16. The nucleic acid of claim 15, wherein the 2 ribosome binding sites are 2 different ribosome binding sites.

17. The nucleic acid of any one of claims 1-16, wherein the nucleic acid comprises a modified nucleic acid.

18. The nucleic acid of claim 17, wherein the modification comprises at least one locked nucleic acid residue.

19. The nucleic acid of claim 17 or 18, wherein the modification comprises at least one phosphorothioate linkage.

20. The nucleic acid of any one of claims 17-19, wherein the modification comprises an ethylene bridged nucleotide, a peptide nucleic acid, a phosphorodiamidate morpholino, a 5′-Vinyl-phosphonate, or combinations thereof.

21. The nucleic acid of any one of claims 1-20, wherein the targeting region comprises at least 12 nucleotides.

22. The nucleic acid of any one of claims 1-21, wherein the targeting region is 20-50 nucleotides.

23. The nucleic acid of any one of claims 1-22, wherein the translational activating region is 5′ of the targeting region.

24. The nucleic acid of any one of claims 1-22, wherein the translational activating region is 3′ of the targeting region.

25. The nucleic acid of any one of claims 1-24, wherein the translation factor comprises eIF3 or eIF4G.

26. The nucleic acid of any one of claims 1-25, wherein the targeting region is complementary to at least a portion of a 3′UTR region of the mRNA.

27. The nucleic acid of any one of claims 1-25, wherein the targeting region is complementary to at least a portion of a 5′UTR region of the mRNA.

28. The nucleic acid of any one of claims 1-25, wherein the targeting region is complementary to at least a portion of the coding region of the mRNA.

29. The nucleic acid of any one of claims 1-28, wherein the targeting region comprises a region that is complementary to a tumor suppressor mRNA.

30. The nucleic acid of claim 29, wherein the tumor suppressor comprises PTEN.

31. The nucleic acid of any one of claims 1-28, wherein the targeting region comprises a region that is complementary to a mRNA from the SYNGAP1, ATP1A3, SCN1A, SCN2, or SIM1 gene.

32. The nucleic acid of any one of claims 1-31, wherein the nucleic acid comprises deoxyribonucleic acid (DNA).

33. The nucleic acid of any one of claims 1-30, wherein the nucleic acid is ribonucleic acid (RNA).

34. A cDNA of the DNA of claim 32 or of the RNA of claim 33.

35. A vector comprising the cDNA of claim 34.

36. A host cell comprising the nucleic acid of any one of claims 1-33, the cDNA of claim 34, or the vector of claim 35.

37. The host cell of claim 36, wherein the host cell is a bacterial cell or a mammalian cell.

38. The host cell of claim 37, wherein the host cell comprises a human cell.

39. A method for increasing translation of a target mRNA in a cell comprising administering the nucleic acid of any one of claims 1-33, the cDNA of claim 34, or the vector of claim 35 to the cell, wherein the target region of the nucleic acid is complementary to the target mRNA.

40. A method for treating a haploinsufficiency disorder in a subject, wherein the haploinsufficiency disorder is further defined as a deficiency in the protein expression of one or both alleles of a target gene, the method comprising administering the nucleic acid of any one of claims 1-33, the cDNA of claim 34, the vector of claim 35, or the cell of claim 36-38 to the subject, wherein the target region of the nucleic acid is complementary to a mRNA transcribed from the target gene.

41. The method of claim 40, wherein the subject has one allele of the target gene that encodes a wild type or functional protein and one variant allele of the target gene.

42. The method of claim 41, wherein the variant allele of the target gene comprises a complete or partial loss of function mutation.

43. The method of any one of claim 41 or 42, wherein the target region is complementary to the mRNA transcribed from the wild type or functional allele of the gene.

44. The method of any one of claims 39-43, wherein the target mRNA encodes for Peptidylprolyl Isomerase B (PPIB) or wherein the target gene comprises PPIB.

45. The method of any one of claims 39-43, wherein the target mRNA encodes for a cell cycle inhibitor or wherein the target gene comprises a cell cycle inhibitor gene.

46. The method of any one of claims 40-43, wherein the haploinsufficiency disorder comprises Wolfram syndrome.

47. The method of claim 46, wherein the target gene comprises Wolfram syndrome 1 (WFS1).

48. The method of any one of claims 40-43, wherein the haploinsufficiency disorder comprises Alzheimer's Disease.

49. The method of claim 48, wherein the target gene comprises ATP binding cassette subfamily A member 7 (ABCA7).

50. A method for treating cancer in a subject comprising administering the nucleic acid of claim 29 or 30 to the subject.

51. The method of claim 50, wherein the cancer comprises breast cancer.

52. The method of claim 51, wherein the breast cancer comprises triple negative breast cancer (TNBC).

53. The method of any one of claims 50-52, wherein the method further comprises administration of a PI3K/mTOR inhibitor.

54. The method of claim 53, wherein the inhibitor comprises BEZ235.

55. The method of any one of claims 50-54, wherein the subject is or has been determined to have a cancer that is resistant to PI3K/mTOR inhibition.

56. A method for treating a disease in a subject, comprising administering the nucleic acid of any one of claims 1-33, the cDNA of claim 34, the vector of claim 35, or the cell of claim 36-38.

57. A chimeric RNA comprising a targeting region and a translational activating region, wherein the translational activating region comprises an IRES or a ribosome and/or translation factor binding fragment thereof and wherein the targeting region comprises an antisense RNA that is complementary to a target mRNA.

58. A method for increasing translation of a target mRNA in a cell comprising administering a chimeric RNA comprising a targeting region and a translational activating region, wherein the translational activating region comprises an IRES or a ribosome and/or translation factor binding fragment thereof and wherein the targeting region comprises an antisense RNA that is complementary to a target mRNA.
